(19) United States
(12) Patent Application Publication
(10) Pub. No.: US 2026/0241045 A1
(43) Pub. Date: Aug. 20, 2026

(54) MACROCYCLIC COMPLEXES OF RADIONUCLIDES AND USES THEREOF

(71) Applicant: UT-Battelle, LLC, Oak Ridge, TN (US)

(72) Inventors: Nikki Thiele, Kingston, TN (US); Megan Simms, Knoxville, TN (US); Md Faizul Islam, Knoxville, TN (US)

(21) Appl. No.: 19/050,255

(22) Filed: Feb. 11, 2025

Related U.S. Application Data
(60) Provisional application No. 63/552,212, filed on Feb. 12, 2024.

(51) Int. Cl. A61K 51/04; C07D 471/18

(52) U.S. Cl. CPC A61K 51/0482; C07D 471/18

(57) ABSTRACT

A macrocycle-radionuclide complex comprising the following formula:

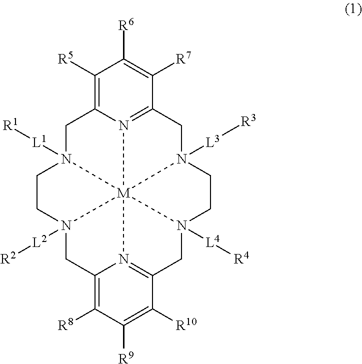

wherein: L¹, L², L³, and L⁴ are linkers; R¹, R², R³, and R⁴ are independently selected from: (i) oxygen-containing groups; (ii) aryl groups; and (iii) heteroaryl groups; R⁵, R⁶, R⁷, R⁸, R⁹, and R¹⁰ are independently selected from: (i) H; (ii) hydrophilic groups; and (iii) highly reactive groups useful in attaching the complex to a cancer cell targeting agent; and M is a radionuclide atom useful in cancer therapy or diagnostic imaging. Also described is a method of treating cancer in a subject by administering to the subject a pharmaceutically effective amount of the composition described above. Also described is a method of imaging biological tissue in a subject by administering the composition to the subject and imaging biological tissue in the subject by a nuclear medicine imaging technique.

### CROSS REFERENCE TO RELATED APPLICATION

The present application claims benefit of U.S. Provisional Application No. 63/552,212, filed on Feb. 12, 2024, all of the contents of which are incorporated herein by reference.

### STATEMENT REGARDING FEDERALLY SPONSORED RESEARCH

This invention was made with government support under Prime Contract No. DE-AC05-000R22725 awarded by the U.S. Department of Energy. The government has certain rights in the invention.

### FIELD OF THE INVENTION

The present invention generally relates to biocompatible radionuclide compositions useful in radiopharmaceutical and cancer treatment therapy. The present invention more particularly relates to methods of treatment and imaging of biological tissue using such compositions, particularly chelated compositions containing one or more therapeutic or imaging radionuclides. The present invention further relates to methods for selective targeting of biocompatible radionuclide compositions to specific biological tissue, such as tumor cells.

### BACKGROUND

Radioactive isotopes of metal ions across the periodic table, from alkalis to actinides, are increasingly being explored in the clinic to diagnose and treat cancer. The use of radiometals in this context leverages the unique ionizing emissions afforded by nuclear decay. Radiometals that emit positrons (β⁺) or gamma (γ) rays can be used to image lesions via positron emission tomography (PET) or single-photon emission computed tomography (SPECT), respectively. By contrast, therapy can be achieved using radiometals that emit cytotoxic alpha (α) particles, beta (β) particles, or Meitner-Auger electrons. When paired together, diagnostic and therapeutic radiometals with complementary properties can form powerful “theranostic” platforms to provide precise information about disease localization, dosimetry, and treatment response. Consequently, this information can be harnessed to develop treatment plans tailored to the individual patient.

Irrespective of emission type, a universal requirement for the implementation of radioactive metal ions in targeted nuclear medicine is their attachment to tumor-seeking carrier molecules, such as antibodies or peptides, via bifunctional chelators. This attachment ensures that each radiometal is selectively delivered to sites of disease, minimizing off-target radiotoxicity to healthy organs. In this regard, the formation of metal-chelator complexes of high thermodynamic and kinetic stability is crucial to prevent dissociation in vivo. To achieve this stability, many ligand design efforts have focused on matching and optimizing the structure of a chelator to the coordination preferences of a single radiometal ion of interest. This strategy has enabled significant advances over the last decade in the development of new metal-based radiopharmaceuticals. However, as the number and chemical diversity of medically useful radiometals continues to expand, ligand development using the conventional one-metal-one-chelator paradigm has struggled to keep pace. With a shortage of suitable chelators for the ever-growing list of radiometals, the advancement of many emerging radiometals has been halted in the so-called “valley of death” between production and clinical implementation, unable to reach their full medicinal potential.

To address this challenge, new efforts have been directed towards developing chelators for nuclear medicine that exhibit more versatile binding properties. For example, the 18-membered macrocycles macrodipa, py-macrodipa, and py₂-macrodipa were developed recently as dual-size-selective chelators for binding both large and small radioactive rare-earth ions, such as the Meitner-Auger-electron emitter lanthanum-135 (¹³⁵La³⁺, 8-coordinate ionic radius=1.16 Å) and the β⁺ emitter scandium-44 (⁴⁴Sc³⁺, 8-coordinate ionic radius=0.87 Å), respectively.

With respect to metal-binding range, however, the 12-membered tetraaza macrocycle DOTA remains unsurpassed in its ability to stabilize a wide assortment of radiometals with disparate chemical and physical properties. DOTA plays a central role in theranostics as the bifunctional chelator in several clinically approved gallium-68 (⁶⁸Ga) and ¹⁷⁷Lu radiopharmaceuticals for PET imaging and targeted β⁻ therapy of cancer, respectively (e.g., U. Hennrich and K. Kopka, Pharmaceuticals, 2019, 12, 114; U. Hennrich and M. Benešová, Pharmaceuticals, 2020, 13, 38; and U. Hennrich and M. Eder, Pharmaceuticals, 2022, 15, 1292). Targeted constructs containing DOTA are also being investigated in clinical trials for use with copper-64 (⁶⁴Cu) for PET and with ²²⁵Ac and other a emitters for targeted a therapy (TAT). However, despite its demonstrated versatility, one prominent shortcoming of DOTA is its inability to chelate most radiometals at room temperature. Although its slow complex formation kinetics can be overcome by heating these radiolabeling reactions, high temperatures are not compatible with the use of thermolabile biological targeting vectors, such as antibodies.

As such, the development of alternative chelators that retain DOTA's broad chelation properties but radiolabel rapidly at low temperature would be highly valuable for use in nuclear medicine. Also, there would be a substantial advantage for peptide or antibody conjugates to use a single chelator for both imaging and therapy because it would have nearly identical pharmacokinetic properties and would simplify regulatory filing, as only a single chemical entity would need to be advanced to production, quality control, and clinical evaluation. However, chelators having such advantageous properties have remained elusive.

### SUMMARY

In one aspect, the present disclosure is directed to biocompatible macrocyclic radionuclide complexes that are thermodynamically and kinetically stable for a wide range of radionuclides, thus advantageously circumventing the conventional one-metal-one-chelator paradigm that has long hindered the development and production of radionuclide complexes for medical use. The present invention has achieved this by use of novel chelators that have herein been found to be highly versatile in their ability to form stable complexes with a wide number of different types of radionuclides. The chelators described herein are further advantageous by being amenable to the inclusion of reactive groups that can be used to bind them to targeting agents. The targeting agents are typically those useful in the targeting of cancer cells.

More particularly, the present disclosure is directed to macrocyclic radionuclide complexes within the following generic formula:

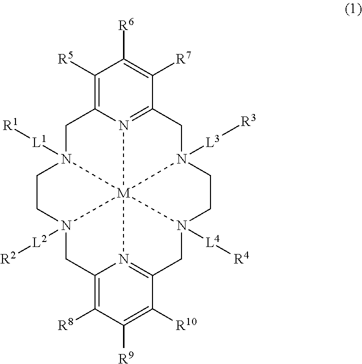

In Formula (1), L¹, L², L³, and L⁴ are independently selected from a linker of the formula (CH₂)ₚ, wherein p is an integer of 0, 1, 2, or 3; R¹, R², R³, and R⁴ are independently selected from: (i) oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt; (ii) aryl groups; and (iii) heteroaryl groups; R⁵, R⁶, R⁷, R⁸, R⁹, and R¹⁰ are independently selected from: (i) H; (ii) hydrophilic groups of the formula L^{a}_{z}-R^{a}, wherein L^{a} is a hydrocarbon linker containing 1-12 carbon atoms, z is 0 or 1, and R^{a} is independently selected from the group consisting of —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂; and (iii) highly reactive groups of the formula L^{b}_{z}-R^{b}, wherein L^{b} is a hydrocarbon linker containing 1-12 carbon atoms and optionally containing one or more heteroatoms selected from O, N, and S; z is 0 or 1; and R^{b} is independently selected from the group consisting of —OCN, —SCN, —NCO, —NCS, —NR′—NR′₂, —NR′₂, —N═C═N—R′, —SH, —SSR′, —SO₂Cl, —C(O)Cl, alkyne-containing groups, alkene-containing groups, azide-containing groups, aldehyde-containing groups, tetrazine-containing groups, activated ester groups, tetrafluorophenyl, maleimide groups, and epoxide groups; and M is a radionuclide atom useful in diagnostic imaging or cancer therapy.

In another aspect, the present disclosure is directed to chelator molecules, such as shown in Formula (1), but which contain one or more functional groups (e.g., hydrocarbon groups, oxygen-containing groups, or highly reactive groups) on the pyridine ring. The functionalized chelator molecules are within the following generic formula:

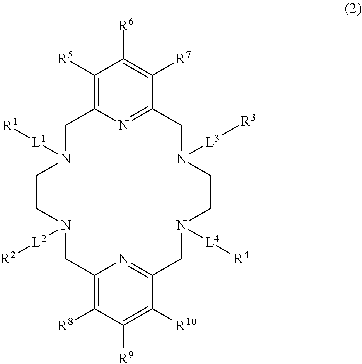

In Formula (2), L¹, L², L³, L⁴, R¹, R², R³, R⁴, R⁵, R⁶, R⁷, R⁸, R⁹, and R¹⁰ are as defined under Formula (1), except that at least one of R⁵, R⁶, R⁷, R⁸, R⁹, and R¹⁰ (groups on the pyridine rings) is a non-H group selected from groups (ii) and (iii).

In another aspect, the present disclosure is directed to cancer cell-targeting macrocycle-radionuclide complexes within the following generic formula:

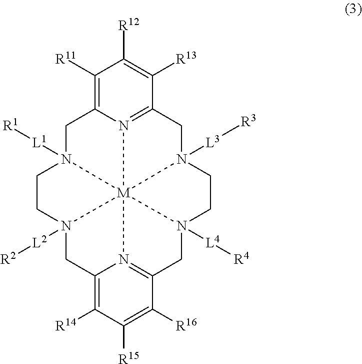

In Formula (3), L¹, L², L³, and L⁴ are independently selected from a linker of the formula (CH₂)ₚ, wherein p is an integer of 0, 1, 2, or 3; R¹, R², R³, and R⁴ are independently selected from: (i) oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt; (ii) aryl groups; and (iii) heteroaryl groups; R¹¹, R¹², R¹³, R¹⁴, R¹⁵, and R¹⁶ are independently selected from: (i) H; (ii) hydrophilic groups of the formula L^{a}_{z}-R^{a}, wherein L^{a} is a hydrocarbon linker containing 1-12 carbon atoms, z is 0 or 1, and R^{a} is independently selected from the group consisting of —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂; and (iii) cancer cell targeting groups of the formula L^{c}_{z}-R^{c}, wherein L^{c} is a hydrocarbon linker containing 1-12 carbon atoms and optionally containing one or more heteroatoms selected from O, N, and S, or L^{c} is a linking particle; z is 0 or 1; and R^{c} is a cancer cell targeting agent; wherein at least one of R¹¹, R¹², R¹³, R¹⁴, R¹⁵, and R¹⁶ is a cancer cell targeting group L^{c}_{z}-R^{c}; and M is a radionuclide atom useful in diagnostic imaging or cancer therapy.

In another aspect, the present disclosure is directed to a method of treating cancer in a subject, the method comprising administering to the subject a pharmaceutically effective amount of any of the radionuclide complexes, such as in Formula (1) or (3), as described above. The cancer may be, for example, breast cancer, prostate cancer, ovarian cancer, leukemia, colorectal cancer, lymphoma, or neuroblastoma or any of the others mentioned in this disclosure. In some embodiments, the complex is attached to a cancer cell targeting group capable of selectively transporting and attaching the composition to cancer cells when introduced into an organism.

In another aspect, the present disclosure is directed to a method of imaging biological tissue in a subject, the method comprising administering to the subject a pharmaceutically effective amount of any of the radionuclide complexes, such as in Formula (1) or (3), as described above, followed by imaging of the biological tissue. The imaging is typically done on cancerous tissue. Typically, the complex is attached to a cancer cell targeting group capable of selectively transporting and attaching the composition to cancer cells when introduced into an organism. Biological tissue that has been selectively targeted by the complex is then imaged. The imaging is typically made possible by a nuclear medicine imaging technique.

### BRIEF DESCRIPTION OF THE DRAWINGS

FIG. 1 shows X-ray crystal structures of Sc³⁺ (left) and In³⁺ (right) complexes of PYTA. Ellipsoids for carbons and heteroatoms are drawn at the 50% probability level. Counteranions, outer-sphere solvent molecules, and hydrogen atoms attached to carbon centers have been omitted for the sake of clarity.

FIG. 2 shows configurations of the macrocyclic backbone of PYTA when bound to La³⁺, Lu³⁺, In³⁺, Sc³⁺, or Cu²⁺. 8-Coordinate ionic radii (6-coordinate for Cu²⁺) are provided below each metal ion. Pendant acetate arms have been omitted for clarity.

FIGS. 3a-3d are graphs showing concentration-dependent radiolabeling of PYTA, macropa, and DOTA. FIG. 3a shows results for ²²⁵Ac³⁺ (9.3-11.1 kBq); FIG. 3b shows results for [¹⁷⁷Lu]Lu³⁺ (22-37 kBq); FIG. 3c shows results for [¹¹¹In]In³⁺ (74-111 kBq); and FIG. 3d shows results [⁴⁴Sc]Sc³⁺ (185 kBq) after 5 min reaction time. All reactions were conducted at room temperature in NH₄OAc (0.5 M, pH 6.0) except for [⁴⁴Sc]Sc-DOTA, which was at 80° C., V_{T}=150 μL. The error bars represent the standard deviations. Data for ²²⁵Ac-macropa and ²²⁵Ac-DOTA were taken from A. Hu et al., Inorg. Chem., 2022, 61, 12847-12855.

FIGS. 4a-4c show PET imaging and ex vivo biodistribution of [⁴⁴Sc]Sc-PYTA in female Swiss Webster mice (6-8 weeks, n=4 per time point) following intravenous injection. FIG. 4a shows representative 3D maximum intensity projections of the PET acquisitions at 0.1, 4, 35, and 55 min post injection indicating rapid transit from the blood pool to the kidneys and bladder. FIG. 4b is a graph showing the biodistribution of [⁴⁴Sc]Sc-PYTA for select organs after 15 min, 1 h, and 4 h post injection. FIG. 4c is a graph comparing the biodistribution of [⁴⁴Sc]Sc-PYTA and [⁴⁴Sc]ScCl₃ at 1 h post injection. Values for each time point are given as the mean % ID per g±1 SD. Data for [⁴⁴Sc]ScCl₃ are taken from N. Benabdallah et al., EJNMMI Res., 2023, 13, 17.

FIG. 5 is a graph showing ex vivo biodistribution of ²²⁵Ac-PYTA for select organs following intravenous injection in mice. Female Swiss Webster mice (6-8 weeks) were sacrificed 15 min, 1 h, 24 h, or 48 h post injection. Values for each time point are given as the mean % ID per g±1 SD (n=4).

### DETAILED DESCRIPTION

As used herein, the term “hydrocarbon group” (also denoted by the group R) is defined as a chemical group containing at least carbon and hydrogen atoms. In some embodiments, R is composed of solely carbon and hydrogen. In other embodiments, R is composed of carbon and hydrogen along with one or more heteroatoms, such as one or more of halogen (e.g., fluorine, chlorine, or bromine), oxygen, nitrogen, sulfur, boron, silicon, and/or phosphorus atoms. In different embodiments, one or more of the hydrocarbon groups contain, for example, 1, 2, 3, 4, 5, 6, 7, 8, 9, 10, 11, or 12 carbon atoms, or a number of carbon atoms within a particular range bounded by any two of the foregoing carbon numbers (e.g., 1-12, 1-8, 1-6, 1-5, 1-4, 1-3, 2-12, 2-8, 2-6, 2-5, 2-4, or 2-3 carbon atoms). Hydrocarbon groups in different compounds described herein, or in different generic groups of a compound, may possess the same or different number (or preferred range thereof) of carbon atoms. For example, any one of R¹, R², R³, and/or R⁴ in any of the generic formulas disclosed herein may independently contain a number of carbon atoms within any of the ranges provided above.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, and n-dodecyl groups.

In a second set of embodiments, the hydrocarbon group (R) is saturated and branched, i.e., a branched alkyl group. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl groups, isoheptyl, isooctyl, and the numerous other branched alkyl groups having up to 12 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), 3-butynyl, and the numerous other straight-chained alkenyl or alkynyl groups having up to 12 carbon atoms.

In a fifth set of embodiments, the hydrocarbon group (R) is unsaturated and branched, i.e., a branched olefinic or alkenyl group. Some examples of branched olefinic groups include propen-2-yl (CH₂═C·—CH₃), 1-buten-2-yl (CH₂═C·—CH₂—CH₃), 1-buten-3-yl (CH₂═CH—CH·—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 12 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group can be aromatic (aryl) or aliphatic.

Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, azulene, indene, or heptalene fused ring systems. All of the foregoing cyclic groups are carbocyclic groups. However, the cyclic groups may contain one or more ring heteroatoms selected from N, O, and S atoms to result in a heteroaromatic ring or ring system, e.g., pyridinyl, imidazolyl, piperazinyl, and pyrrolidinyl groups.

The hydrocarbon group may also be a result of one type of hydrocarbon group attached to another type of hydrocarbon group. An example of such a group is an alkyl group attached to an aryl group, which may be referred to as an aralkyl group. Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Aralkyl groups may be substituted or unsubstituted. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups, such as 4-indanylethyl. Representative substituted aralkyl groups may be substituted one or more times with substituents, such as those listed above.

As noted earlier above, any of the hydrocarbon groups exemplified herein may include one or more heteroatoms. More particularly, the hydrocarbon group may be substituted with one or more heteroatoms, which are atoms other than carbon and hydrogen, examples of which have been provided above. The term “substituted”, as used herein, generally refers to a hydrocarbon group in which a bond to a hydrogen atom contained therein is replaced by a bond to a non-hydrogen non-carbon atom. Substituted groups also include groups in which one or more bonds to a carbon or hydrogen atom(s) are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include, but are not limited to: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF5), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups, such as substituted cycloalkyl, aryl, heterocyclyl and heteroaryl groups, also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as provided above.

Any of the hydrocarbon groups provided above may or may not be substituted by one or more heteroatom-containing groups. Some examples of oxygen-containing groups include hydroxy (OH), alkoxy (ORS), carbonyl-containing (e.g., carboxylic acid, ketone, aldehyde, carboxylic ester, amide, and urea functionalities), nitro (NO₂), carbon-oxygen-carbon (ether), sulfonyl, and sulfinyl (i.e., sulfoxide) groups, wherein R^{s} is typically a hydrocarbon group that includes 1-6 (or 1-3) carbon atoms. Some particular examples of alkoxy groups —OR include methoxy, ethoxy, n-propoxy, isopropoxy, n-butoxy, isobutoxy, t-butoxy, phenoxy, benzyloxy, 2-hydroxyethoxy, 2-methoxyethoxy, 2-ethoxyethoxy, vinyloxy, and allyloxy groups. In the case of an ether group, the ether group can also be a polyalkyleneoxide (polyalkyleneglycol) group, such as a polyethyleneoxide group. Some examples of nitrogen-containing groups include primary amine, secondary amine, tertiary amine (i.e., —NR₂ or NR₃+, wherein R is independently selected from H and hydrocarbon groups set forth above), nitrile, amide (i.e., —C(O)NR₂ or —NRC(O)R, wherein R is independently selected from hydrogen atom and hydrocarbon groups set forth above), imine (e.g., —CR═NR, wherein R is independently H or a hydrocarbon group), oxime (—CR═N—OH), amidoxime (—C(NH₂)═N—OH), nitro, urea (—NR—C(O)—NR₂, wherein R is independently H or a hydrocarbon group), and carbamate groups (—NR—C(O)—OR, wherein R is independently H or a hydrocarbon group). Some examples of phosphorus-containing groups include —PR₂, —PR₃⁺, —P(═O)R₂, —P(OR)₂, —O—P(OR)₂, —R—P(OR)₂, —P(═O)(OR)₂, —O—P(═O)(OR)₂, —O—P(═O)(OR)(R), —O—P(═O)R₂, —R—P(═O)(OR)₂, —R—P(═O)(OR)(R), and —R—P(═O)R₂ groups, wherein R is independently selected from hydrogen atom and hydrocarbon groups set forth above. Some examples of sulfur-containing groups include mercapto (i.e., —SH), thioether (i.e., sulfide, e.g., —SR), disulfide (—R—S—S—R), sulfoxide (—S(O)R), sulfone (—SO₂R), sulfonate (—S(═O)₂OR, wherein R is H, a hydrocarbon group, or a cationic group), and sulfate groups (—OS(═O)₂OR, wherein R is H, a hydrocarbon group, or a cationic group). Some examples of halide atoms include fluorine, chlorine, bromine, and iodine.

One or more of the heteroatoms described above (e.g., oxygen, nitrogen, and/or sulfur atoms) can be inserted between carbon atoms (e.g., as —O—, —NH—, or —S—) in any of the hydrocarbon groups described above to form a heteroatom-substituted hydrocarbon group. Alternatively, or in addition, one or more of the heteroatom-containing groups can replace one or more hydrogen atoms on the hydrocarbon group. In some embodiments, one or more of the above described heteroatoms or heteroatom-containing groups are excluded from R or a linker thereof.

In some embodiments, the hydrocarbon group is or includes a cyclic or polycyclic group that includes at least one ring heteroatom (for example, one, two, three, four, or higher number of heteroatoms). Such ring heteroatom-substituted cyclic groups are referred to herein as “heterocyclic” or “heterocyclyl” groups. As used herein, a “ring heteroatom” is an atom other than carbon and hydrogen (typically, selected from nitrogen, oxygen, and sulfur) that is inserted into, or replaces a ring carbon atom in, a hydrocarbon ring structure. In some embodiments, the heterocyclic group is saturated, while in other embodiments, the heterocyclic group is unsaturated (i.e., aliphatic or aromatic heterocyclic groups, wherein the aromatic heterocyclic group is also referred to herein as a “heteroaromatic ring,” “heteroaryl ring,” or a “heteroaromatic/heteroaryl fused-ring system” in the case of at least two fused rings, at least one of which contains at least one ring heteroatom). In some embodiments, the heterocyclic group is bound via one of its ring carbon atoms to another group (i.e., other than hydrogen atom and adjacent ring atoms), while the one or more ring heteroatoms are not bound to another group. In other embodiments, the heterocyclic group is bound via one of its heteroatoms to another group, while ring carbon atoms may or may not be bound to another group.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged poly cyclic ring systems containing a heteroatom such as, but not limited to, quinuclidyl.

Some examples of saturated heterocyclic groups include those containing at least one oxygen atom (e.g., oxetane, tetrahydrofuran, tetrahydropyran, 1,4-dioxane, 1,3-dioxane, and 1,3-dioxepane rings), those containing at least one nitrogen atom (e.g., pyrrolidine, piperidine, piperazine, imidazolidine, azepane, and decahydroquinoline rings), those containing at least one sulfur atom (e.g., tetrahydrothiophene, tetrahydrothiopyran, 1,4-dithiane, 1,3-dithiane, and 1,3-dithiolane rings), those containing at least one oxygen atom and at least one nitrogen atom (e.g., morpholine and oxazolidine rings), those containing at least one oxygen atom and at least one sulfur atom (e.g., 1,4-thioxane), and those containing at least one nitrogen atom and at least one sulfur atom (e.g., thiazolidine and thiamorpholine rings).

Some examples of unsaturated heterocyclic groups include those containing at least one oxygen atom (e.g., furan, pyran, 1,4-dioxin, and dibenzodioxin rings), those containing at least one nitrogen atom (e.g., pyrrole, imidazole, pyrazole, pyridine, pyrazine, pyrimidine, 1,3,5-triazine, azepine, diazepine, indole, purine, benzimidazole, indazole, 2,2′-bipyridine, quinoline, isoquinoline, phenanthroline, quinoxaline, quinazoline, pyridazine, and cinnoline), those containing at least one sulfur atom (e.g., thiophene, thianaphthene, and benzothiophene rings), those containing at least one oxygen atom and at least one nitrogen atom (e.g., oxazole, isoxazole, benzoxazole, benzisoxazole, and oxazoline rings), and those containing at least one nitrogen atom and at least one sulfur atom (e.g., thiazole, isothiazole, benzothiazole, benzoisothiazole, and thiazoline rings).

Heterocyclyl groups may be substituted or unsubstituted. Some further examples of heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidinyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Some further examples of heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Heteroaryl groups may be substituted or unsubstituted. Thus, the phrase “heteroaryl groups” includes fused ring compounds as well as includes heteroaryl groups that have other groups bonded to one of the ring members, such as alkyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene. Such groups may further be substituted or unsubstituted.

Alkoxy groups are hydroxyl groups (—OH) in which the bond to the hydrogen atom is replaced by a bond to a carbon atom of a substituted or unsubstituted alkyl group as defined above. Alkoxy groups may be denoted as OR groups, where R may be any of the hydrocarbon groups described above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Alkoxy groups may be substituted or unsubstituted. Representative substituted alkoxy groups may be substituted one or more times with substituents such as those listed above.

The terms “alkanoyl” and “alkanoyloxy,” as used herein, can refer, respectively, to —C(O)-alkyl and —O—C(O)-alkyl groups, where in some embodiments the alkanoyl or alkanoyloxy groups each contain 2-5 carbon atoms. Similarly, the terms “aryloyl” and “aryloyloxy” respectively refer to —C(O)-aryl and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylic acid” as used herein refers to a compound with a —C(O)OH group. The term “carboxylate” as used herein refers to a —C(O)O— or —C(O)OR group. A “protected carboxylate” refers to an —C(O)O-G ester group where G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein. Moreover, G may be an ester activating group (e.g., carbodiimides or NHS), as well known in the art.

The term “amide” (or “amido”) includes C- and N-amide groups, i.e., —C(O)NRR, and —NRC(O)R groups, respectively. The R groups are each independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NRC(O)—(C₁₋₅ alkyl) and the group is termed “carbonylamino,” and in others the amide is —NHC(O)-alkyl and the group is termed “alkanoylamino.”

The term “amine” (or “amino”) as used herein refers to —NRR groups, wherein the R groups are each independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is H2, methylamino, dimethylamino, ethylamino, diethylamino, propylamino, isopropylamino, phenylamino, or benzylamino. The term “sulfonamido” includes S- and N-sulfonamide groups. The term “thiol” refers to —SH groups, while sulfides include —SR groups. The term “halogen” or “halo” as used herein refers to bromine, chlorine, fluorine, or iodine. In some embodiments, the halogen is fluorine. In other embodiments, the halogen is chlorine or bromine. The term “hydroxyl” as used herein can refer to —OH or its ionized form. The term “isocyano” refers to —NC. The term “isothiocyano” refers to —NCS.

All ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes at least each individual integer. Thus, for example, a group having 1-3 atoms refers to groups having 1, 2, or 3 atoms. Similarly, a group having 1-5 atoms refers to groups having 1, 2, 3, 4, or 5 atoms, and so forth.

Notably, the term “hydrocarbon group” also includes “hydrocarbon linkers.” Hydrocarbon linkers may be denoted by “L,” such as L¹ or L², as depicted in Formula (1). A hydrocarbon linker can be derived from any of the hydrocarbon groups described above by removing a hydrogen atom from the hydrocarbon group. For example, a hydrogen atom may be removed from an ethyl group (—CH₂CH₃) to result in an ethylene (—CH₂CH₂—) linker, or a hydrogen atom may be removed from an ethyleneoxy group (—OCH₂CH₃) to result in an ethyleneoxy (—OCH₂CH₂—) linker. As another example, a hydrogen atom may be removed from a C₆H₅—CH₂CH₂O— group to result in a —C₆H₄—CH₂CH₂O— linker, wherein C₆H₅ represents a phenyl group. As another example, a hydrogen atom may be removed from a C₆H₅—C≡C— group to result in a —C₆H₄—C≡C— linker. Linkers disclosed in this application (e.g., L¹ and R¹) may independently have any of the carbon numbers provided above and may be derived from any of the linear or branched alkyl, alkenyl, or alkynyl groups described above or saturated or unsaturated cyclic groups described above. The linker may also optionally include one or more heteroatoms, as described above. Any linkers disclosed in this application, such as L^{a}, L^{b}, or L^{c}, may independently be selected from any such linkers derived from any of the hydrocarbon groups described above.

In one aspect, the present disclosure is directed to a macrocycle-radionuclide complex having the following formula:

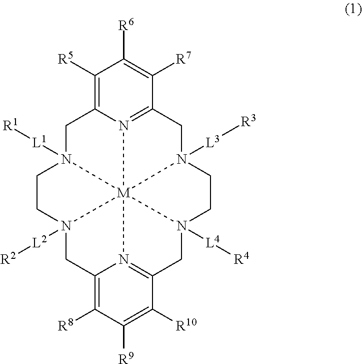

The variables L¹, L², L³, and L⁴ in Formula (1) are independently selected from a linker of the formula (CH₂)ₚ, wherein p is an integer of 0, 1, 2, or 3. In the event that p is 0, no linker is present, and thus, the linker represents a bond connecting the respective R group to the respective N atom shown in Formula (1). Thus, for example, if p in L¹ is 0, then L¹ represents a covalent bond to R¹. In a first set of embodiments, one, two, three, or all of L¹, L², L³, and L⁴ is/are bonds (i.e., where p is 0). In a second set of embodiments, one, two, three, or all of L¹, L², L³, and L⁴ is/are —CH₂— linkers (i.e., where p is 1). In a third set of embodiments, one, two, three, or all of L¹, L², L³, and L⁴ is/are —CH₂CH₂— linkers (i.e., where p is 2). In a fourth set of embodiments, one, two, three, or all of L¹, L², L³, and L⁴ is/are —CH₂CH₂CH₂— linkers (i.e., where p is 3).

The variables R¹, R², R³, and R⁴ are independently selected from hydrophilic (e.g., oxygen-containing) or metal-binding groups. In a first set of embodiments, precisely or at least one, two, three, or all of the variables R¹, R², R³, and R⁴ are oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt. Any of the foregoing oxygen-containing groups may also function as metal-binding groups. In a second set of embodiments, precisely or at least one, two, three, or all of the variables R¹, R², R³, and R⁴ are aryl groups, such as any of those described above. Typically, the aryl groups contain one or more oxygen-containing or metal-binding substituents. In a third set of embodiments, precisely or at least one, two, three, or all of the variables R¹, R², R³, and R⁴ are heteroaryl groups, such as any of those described above. The heteroaryl groups are often metal-binding by virtue of the heteroatom in the ring, or the heteroaryl group may contain one or more oxygen-containing or metal-binding substituents. The R¹, R², R³, and R⁴ variables may be the same or different. In some embodiments, one, two, three, or all of R¹, R², R³, and R⁴ is/are or includes any of the oxygen-containing groups provided above. In more particular embodiments, one, two, three, or all of R¹, R², R³, and R⁴ is/are —COOR′ or —OR′ group(s), or more particularly, —COOH or —OH groups.

The variables R⁵, R⁶, R⁷, R⁸, R⁹, and R¹⁰ in Formula (1) may be selected from H and non-H groups, including any of the groups described above for L¹-R¹, L²-R², L³-R³, and L⁴-R⁴, in which the linker may be present or absent (as a bond). In a first set of embodiments, precisely or at least one, two, three, or all of the variables R⁵, R⁶, R⁷, R⁸, R⁹, and R¹⁰ are hydrogen (H). In a second set of embodiments, precisely or at least one, two, three, or all of the variables R⁵, R⁶, R⁷, R⁸, R⁹, and R¹⁰ are selected from hydrophilic groups of the formula L^{a}_{z}-R^{a}, wherein La is a hydrocarbon linker containing 1-12 carbon atoms, z is 0 or 1 (which indicates absence or presence of L^{a}, respectively), and R^{a} is independently selected from —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂, or R^{a} may be a group containing any of the foregoing hydrophilic groups. In a third set of embodiments, precisely or at least one, two, three, or all of the variables R⁵, R⁶, R⁷, R⁸, R⁹, and R¹⁰ are selected from highly reactive groups of the formula L^{b}_{z}-R^{b}, wherein L^{b} is a hydrocarbon linker containing 1-12 carbon atoms and optionally containing one or more heteroatoms selected from O, N, and S; z is 0 or 1 (which indicates absence or presence of L^{b}, respectively); and R^{b} is independently selected from —OCN, —SCN, —NCO, —NCS, —NR′—NR′₂, —NR₂, —N═C═N—R′, —SH, —SSR′, —SO₂Cl, —C(O)Cl, alkyne-containing groups, alkene-containing groups, azide-containing groups, aldehyde-containing groups, tetrazine-containing groups, activated ester groups, tetrafluorophenyl, maleimide groups, and epoxide groups, or R^{b} may be a group containing any of the foregoing highly reactive groups.

In some embodiments, precisely or at least one, two, or more of R⁵, R⁶, R⁷, R⁸, R⁹, and R¹⁰ is/are a group or groups independently selected from hydrophilic groups and highly reactive groups, as described above. In some embodiments, precisely or at least one, two, or more of R⁵, R⁶, R⁷, R⁸, R⁹, and R¹⁰ is/are a group or groups independently selected from highly reactive groups, as described above. In particular embodiments, precisely or at least one, two, or more of the variables R⁵, R⁶, R⁷, R⁸, R⁹, and R¹⁰ (or more particularly, R⁶ and/or R⁹) is/are selected from —NCS or —NCO groups, wherein the remainder of R⁵, R⁶, R⁷, R⁸, R⁹, and R¹⁰ groups are typically H atoms, although one or more of the remainder of R⁵, R⁶, R⁷, R⁸, R⁹, and R¹⁰ groups may be hydrophilic group(s) provided they are not reactive with the —NCS or —NCO groups. In other embodiments, precisely or at least one, two, or more of the variables R⁵, R⁶, R⁷, R⁸, R⁹, and R¹⁰ (or more particularly, R⁶ and/or R⁹) is/are selected from alkyne-containing, alkene-containing, or azide-containing groups, wherein the remainder of R⁵, R⁶, R⁷, R⁸, R⁹, and R¹⁰ groups are typically H atoms, although one or more of the remainder of R⁵, R⁶, R⁷, R⁸, R⁹, and R¹⁰ groups may be hydrophilic group(s) provided they are not reactive with the alkyne-containing, alkene-containing, or azide-containing groups.

The radionuclide atom (M) in Formula (1) may be any of the radionuclides known in the medical arts to be useful for treating or imaging of a biological organism. The radionuclide atoms are typically selected from alpha emitting, beta emitting, or Auger emitting. Some examples of alpha emitting radionuclides include actinium-225, thorium-227, thorium-226, radium-223, radium-224, lead-212, astatine-211, uranium-230, bismuth-213, and combinations thereof. Some examples of beta emitting radionuclides include lutetium-177, samarium-153, yttrium-90, strontium-89, iodine-131, and combinations thereof. Some examples of Auger emitting radionuclides include indium-111, iodine-125, iodine-123, platinum-193m, platinum-195m, terbium-161, thallium-201, and combinations thereof. Some examples of imaging radionuclides include iodine-131, iodine-124, iodine-123, technetium-99m, thallium-201, indium-111, gallium-67, cerium-134, copper-64, Ba-133m, and combinations thereof.

In typical embodiments, M is selected from any one of the following radionuclides: ¹⁷⁷Lu³⁺, ⁴³Sc³⁺, ⁴⁴Sc³⁺, ⁴⁷Sc³⁺, ⁶⁶Ga³⁺, ⁶⁷Ga³⁺, ⁶⁸Ga³⁺, ⁸⁶Y³⁺, ⁸⁸Y³⁺, ⁹⁰Y³⁺, ⁸⁹Zr⁴⁺, ¹¹¹In³⁺, ^{110m}In³⁺, ^{114m}In³⁺, ¹³⁵La³⁺, ¹³²La³⁺, ¹³⁴La³⁺, ¹³⁴Ce³⁺, ¹³⁷Ce³⁺, ¹⁵²Dy³⁺, ¹⁴⁹Tb³⁺, ¹⁵²Tb³⁺, ¹⁵⁵Tb³⁺, ¹⁶¹Tb³⁺, ²¹³Bi³⁺, ²¹²Bi³⁺, ²²⁷Th⁴⁺, ²²⁶Th⁴⁺, ²²⁵Ac³⁺, ²²⁶Ac³⁺, ²²³Ra²⁺, ²²⁴Ra²⁺, ¹³¹Ba²⁺, ²¹²Pb²⁺, ²¹²Pb⁴⁺, ²⁵⁵Fm³⁺, ²³⁰U⁶⁺, ²³⁰U⁴⁺, ²⁰¹Tl, ⁹⁰Nb, ¹⁴⁹Pm³⁺, ²³⁹Np⁴⁺, ²³⁹Np⁵⁺, ¹¹⁹Sb³⁺, ¹¹⁷Sb³⁺, ¹¹⁹Sb⁵⁺, ¹¹⁷Sb⁵⁺, ^{52g}Mn, ⁶⁴Cu, ^{133m}Ba²⁺, ⁴⁵Ti⁴⁺, ⁵⁵Co, and ^{58m}Co. Any sub-group of the foregoing radionuclides is also considered. For example, in some embodiments, M is selected from any one of the following radionuclides: ²²⁵Ac³⁺, ²²⁶Ac³⁺, ¹⁷⁷Lu³⁺, ¹¹¹In³⁺, ^{110m}In³⁺, ^{114m}In³⁺, ⁴³Sc³⁺, ⁴⁴Sc³⁺, and ⁴⁷Sc³⁺, or a sub-grouping thereof. Notably, any selection of L¹, L², L³, and L⁴ linkers in Formula (1) may be in combination with any selection of R¹, R², R³, and R⁴ groups in Formula (1) in combination with any selection of R⁵, R⁶, R⁷, R⁸, R⁹, and R¹⁰ groups in Formula (1) in combination with any selection of radionuclides (M) to arrive at a particular macrocycle-radionuclide complex according to Formula (1).

In specific embodiments of Formula (1), the compound has the following structure:

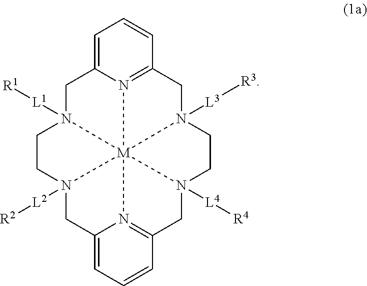

In Formula (1a), L¹, L², L³, L⁴, R¹, R², R³, and R⁴ are as defined under Formula (1) above. Moreover, any selection of L¹, L², L³, and L⁴ linkers in Formula (1a) may be in combination with any selection of R¹, R², R³, and R⁴ groups in Formula (1a) in combination with any selection of radionuclides (M) to arrive at a particular macrocycle-radionuclide complex according to Formula (1a). In particular embodiments, one, two, three, or all of R¹, R², R³, and R⁴ is/are —COOR′ or —OR′ group(s), or more particularly, —COOH or —OH groups. In separate or further embodiments, M may be selected from, more particularly, ²²⁵Ac³⁺, ²²⁶Ac³⁺, ¹⁷⁷Lu³⁺, ¹¹¹In³⁺, ^{110m}In³⁺, ^{114m}In³⁺, ⁴³Sc³⁺, ⁴⁴Sc³⁺, and ⁴⁷Sc³⁺, or a sub-grouping thereof.

In specific embodiments of Formula (1a), the compound has the following structure:

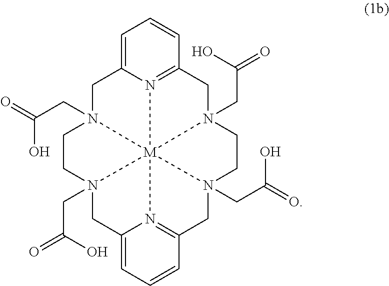

In Formula (1b), M can be any of the radionuclides disclosed earlier above. In some embodiments, M may be selected from, more particularly, ²²⁵Ac³⁺, ²²⁶Ac³⁺, ¹⁷⁷Lu³⁺, ¹¹¹In³⁺, ^{110m}In³⁺, ^{114m}In³⁺, ⁴³Sc³⁺, ⁴⁴Sc³⁺, and ⁴⁷Sc³⁺, or a sub-grouping thereof. The chelator molecule in Formula (1b) is herein also referred to as PYTA. Thus, Formula (1b) encompasses PYTA complexes of any of the possibilities for M provided above.

Some other examples of chelators within the scope of Formula (1) that can bind to a radionuclide (M) include:

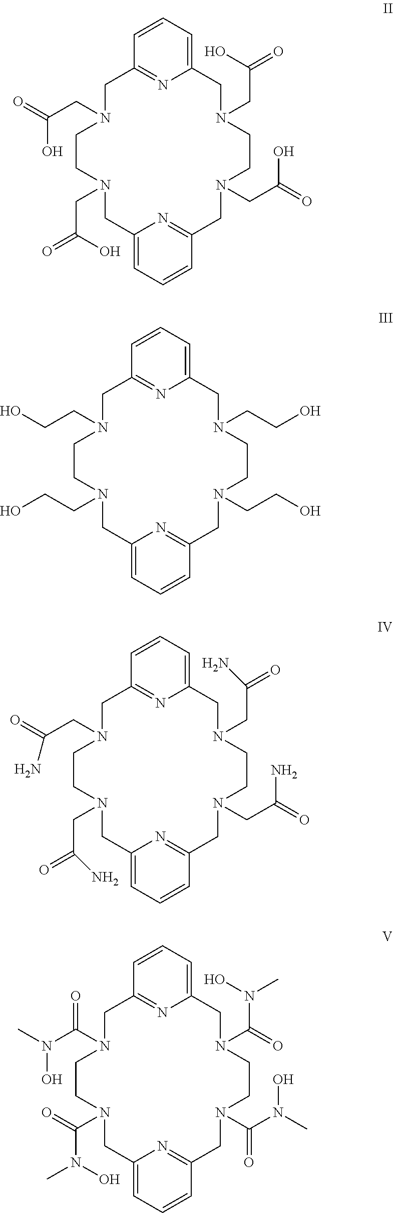

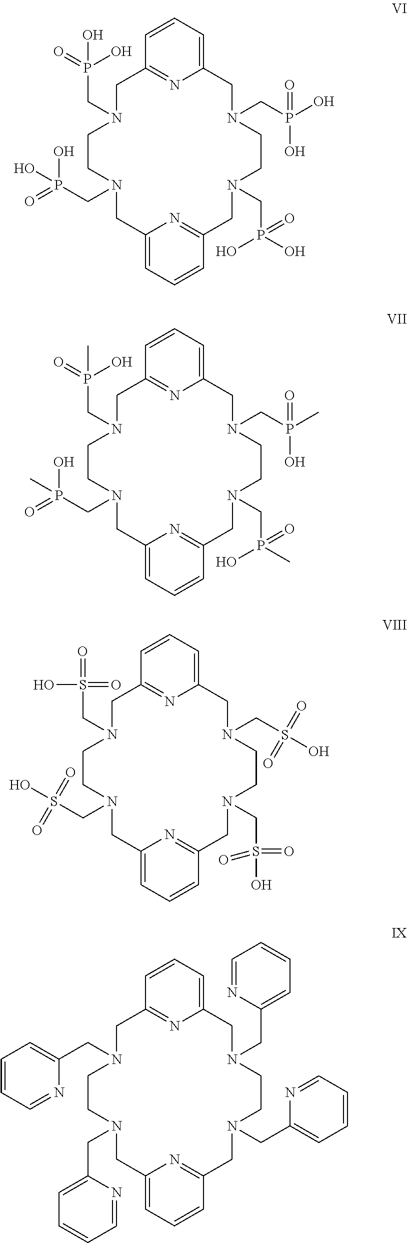

In other embodiments of Formula (1), the compound has the following structure:

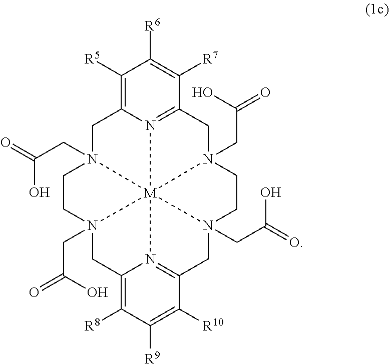

In Formula (1c), precisely or at least one, two, or more of R⁵, R⁶, R⁷, R⁸, R⁹, and R¹⁰ is a group independently selected from hydrophilic groups and highly reactive groups, as defined and provided under Formula (1), wherein the remainder of R⁵, R⁶, R⁷, R⁸, R⁹, and R¹⁰ groups are H atoms. In some embodiments, precisely or at least one, two, or more of R⁵, R⁶, R⁷, R⁸, R⁹, and R¹⁰ is/are independently selected from hydrophilic groups as provided above. In some embodiments, precisely or at least one, two, or more of R⁵, R⁶, R⁷, R⁸, R⁹, and R¹⁰ is/are independently selected from highly reactive groups, as defined and provided under Formula (1). In particular embodiments, precisely or at least one, two, or more of the variables R⁵, R⁶, R⁷, R⁸, R⁹, and R¹⁰ (or more particularly, R⁶ and/or R⁹) is/are selected from —NCS or —NCO groups or with a linker attached to such group, wherein the remainder of R⁵, R⁶, R⁷, R⁸, R⁹, and R¹⁰ groups are typically H atoms, although one or more of the remainder of R⁵, R⁶, R⁷, R⁸, R⁹, and R¹⁰ groups may be hydrophilic group(s) provided they are not reactive with the —NCS or —NCO groups. In other embodiments, precisely or at least one, two, or more of the variables R⁵, R⁶, R⁷, R⁸, R⁹, and R¹⁰ (or more particularly, R⁶ and/or R⁹) is/are selected from alkyne-containing, alkene-containing, or azide-containing groups,
wherein the remainder of R⁵, R⁶, R⁷, R⁸, R⁹, and R¹⁰ groups are typically H atoms, although one or more of the remainder of R⁵, R⁶, R⁷, R⁸, R⁹, and R¹⁰ groups may be hydrophilic group(s) provided they are not reactive with the alkyne-containing, alkene-containing, or azide-containing groups.

Moreover, in typical embodiments, M in Formula (1c) is selected from any one of the following radionuclides: ¹⁷⁷Lu³⁺, ⁴³Sc³⁺, ⁴⁴Sc³⁺, ⁴⁷Sc³⁺, ⁶⁶Ga³⁺, ⁶⁷Ga³⁺, ⁶⁸Ga³⁺, ⁸⁶Y³⁺, ⁸⁸Y³⁺, ⁹⁰Y³⁺, ⁸⁹Zr⁴⁺, ¹¹¹In³⁺, ^{110m}In³⁺, ^{114m}In³⁺, ¹³⁵La³⁺, ¹³²La³⁺, ¹³⁴La³⁺, ¹³⁴Ce³⁺, ¹³⁷Ce³⁺, ¹⁵²Dy³⁺, ¹⁴⁹Tb³⁺, ¹⁵²Tb³⁺, ¹⁵⁵Tb³⁺, ¹⁶¹Tb³⁺, ²¹³Bi³⁺, ²¹²Bi³⁺, ²²⁷Th⁴⁺, ²²⁶Th⁴⁺, ²²⁵Ac³⁺, ²²⁶Ac³⁺, ²²³Ra²⁺, ²²⁴Ra²⁺, ¹³¹Ba²⁺, ²¹²Pb²⁺, ²¹²Pb⁴⁺, ²⁵⁵Fm³⁺, ²³⁰U⁶⁺, ²³⁰U⁴⁺, ²⁰¹Tl, ⁹⁰Nb, ¹⁴⁹Pm³⁺, ²³⁹Np⁴⁺, ²³⁹Np⁵⁺, ¹¹⁹Sb³⁺, ¹¹⁷Sb³⁺, ¹¹⁹Sb⁵⁺, ¹¹⁷Sb⁵⁺, ^{52g}Mn, ⁶⁴Cu, ^{133m}Ba²⁺, ⁴⁵Ti⁴⁺, ⁵⁵Co, and ^{58m}Co. Any sub-group of the foregoing radionuclides is also considered. For example, in some embodiments, M is selected from any one of the following radionuclides: ²²⁵Ac³⁺, ²²⁶Ac³⁺, ¹⁷⁷Lu³⁺, ¹¹¹In³⁺, ^{110m}In³⁺, ^{114m}In³⁺, ⁴³Sc³⁺, ⁴⁴Sc³⁺, and ⁴⁷Sc³⁺, or a sub-grouping thereof.

In another aspect, the present disclosure is directed to chelator molecules bearing a functional group on at least one of the pyridine rings. The functionalized chelators have the following formula:

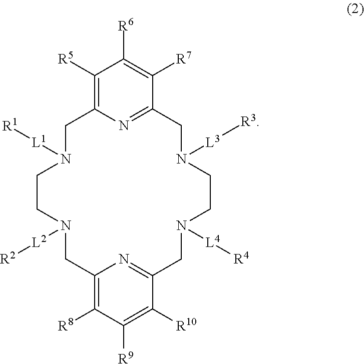

In Formula (2), L¹, L², L³, L⁴, R¹, R², R³, and R⁴ are as defined and exemplified under Formula (1) or (1c), and any selection of these groups can be independently made. In Formula (2), precisely or at least one, two, or more of R⁵, R⁶, R⁷, R⁸, R⁹, and R¹⁰ is a group independently selected from hydrophilic groups and highly reactive groups, as defined and provided under Formula (1), wherein the remainder of R⁵, R⁶, R⁷, R⁸, R⁹, and R¹⁰ groups are H atoms. In some embodiments, precisely or at least one, two, or more of R⁵, R⁶, R⁷, R⁸, R⁹, and R¹⁰ is/are independently selected from hydrophilic groups as provided above. In some embodiments, precisely or at least one, two, or more of R⁵, R⁶, R⁷, R⁸, R⁹, and R¹⁰ is/are independently selected from highly reactive groups, as defined and provided under Formula (1). In particular embodiments, precisely or at least one, two, or more of the variables R⁵, R⁶, R⁷, R⁸, R⁹, and R¹⁰ (or more particularly, R⁶ and/or R⁹) is/are selected from —NCS or —NCO groups or a linker attached to such group, wherein the remainder of R⁵, R⁶, R⁷, R⁸, R⁹, and R¹⁰ groups are typically H atoms, although one or more of the remainder of R⁵, R⁶, R⁷, R⁸, R⁹, and R¹⁰ groups may be hydrophilic group(s) provided they are not reactive with the —NCS or —NCO groups. In other embodiments, precisely or at least one, two, or more of the variables R⁵, R⁶, R⁷, R⁸, R⁹, and R¹⁰ (or more particularly, R⁶ and/or R⁹) is/are selected from alkyne-containing, alkene-containing, or azide-containing groups, wherein the remainder of R⁵, R⁶, R⁷, R⁸, R⁹, and R¹⁰ groups are typically H atoms, although one or more of the remainder of R⁵, R⁶, R⁷, R⁸, R⁹, and R¹⁰ groups may be hydrophilic group(s) provided they are not reactive with the alkyne-containing, alkene-containing, or azide-containing groups.

In specific embodiments of Formula (2), the compound has the following structure:

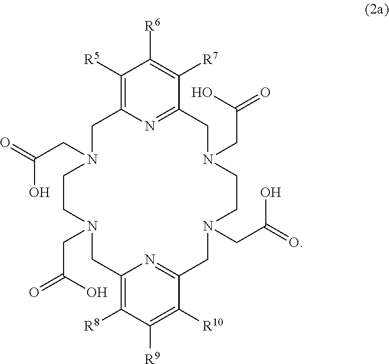

In Formula (2a), R⁵, R⁶, R⁷, R⁸, R⁹, and R¹⁰ are as defined and exemplified under Formulas (1) and (2).

Some examples of chelators within the scope of Formulas (2) and (2a) that can bind to a radionuclide (M) include:

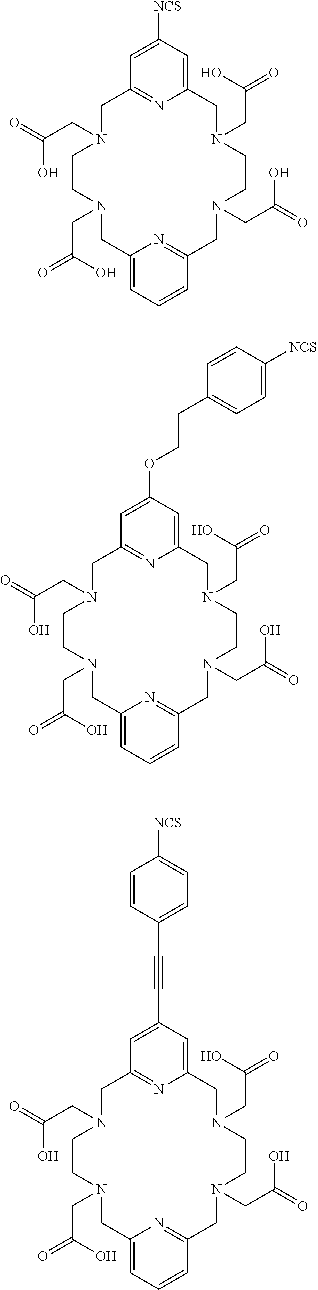

In another aspect, the present disclosure is directed to a cancer cell-targeting radionuclide complex having the following formula:

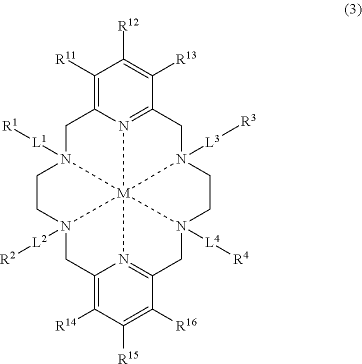

In Formula (3), L¹, L², L³, L⁴, R¹, R², R³, and R⁴ are as defined and exemplified under Formula (1) or (1c), and any selection of these groups can be independently made. The variables R¹¹, R¹², R¹³, R¹⁴, R¹⁵, and R¹⁶ in Formula (3) may be selected from H and non-H groups, including any of the groups described above for L¹-R¹, L²-R², L³-R³, and L⁴-R⁴, in which the linker may be present or absent (as a bond).

In Formula (3), precisely or at least one, two, three, or all of the variables R¹¹, R¹², R¹³, R¹⁴, R¹⁵, and R¹⁶ (or more particularly, R¹² and/or R¹⁵) is/are cancer cell targeting group(s) of the formula L^{c}_{z}-R^{c}, wherein L^{c} is a hydrocarbon linker containing 1-12 carbon atoms and optionally containing one or more heteroatoms selected from O, N, and S, or L^{c} is a linking particle; z is 0 or 1; and R^{c} is a cancer cell targeting agent; wherein at least one of R¹¹, R¹², R¹³, R¹⁴, R¹⁵, and R¹⁶ is a cancer cell targeting group L^{c}_{z}-R^{c}. Notably, in the case where the cancer cell targeting agent is attached to the chelator by reaction of a highly reactive group (e.g., as described under Formula (2)) with a functional group on the cancer cell targeting agent, the linker L^{c} then includes the product of reaction between the highly reactive group and functional group on the cancer cell targeting agent. For example, in the case where the highly reactive group on the chelator is —NCS and the functional group on the cancer cell targeting agent reacting with —NCS is an amine (NH₂), the L^{c} linker includes a —NHC(S)NH— (thiourea) linkage.

Cancer cell targeting agents (R^{c} groups) are well known in the art and include, for example, antibodies (typically monoclonal), antibody fragments, oligopeptides, enzymes, oligonucleotides, DNA vectors, RNA vectors, aptamers, lectins, and small molecule drugs. The cancer cell targeting agents can be any group known in the art capable of selectively targeting cancer cells. By way of example, the selective cancer cell targeting group may target receptor sites specific to cancer cells. The cancer cell targeting group may be composed of amino acids linked by peptide bonds. The selective cancer cell targeting group of any embodiment herein may include a cancer-targeting antibody, antibody fragment, nanobody, a selective targeting oligopeptide containing up to 50 amino acids, an enzyme, a nucleobase-containing moiety (such as an oligonucleotide, DNA or RNA vector, or aptamer), or a lectin. In any embodiment herein, any of the foregoing cancer cell targeting agents may be bound or adsorbed onto a particle (e.g., a nanoparticle or microparticle), with the particle bound to one of the aromatic rings of the macrocyclic composition via a reactive functional group.

The remainder of the R¹¹, R¹², R¹³, R¹⁴, R¹⁵, and R¹⁶ groups (i.e., those that are not L^{c}_{z}-R^{c}, if any) are selected from hydrogen (H) and hydrophilic groups, as described earlier above. In some embodiments, precisely or at least one, two, or three, but not all of the variables R¹¹, R¹², R¹³, R¹⁴, R^{1S}, and R¹⁶ are hydrogen (H). In further or alternative embodiments, precisely or at least one, two, three, but not all of the variables R¹¹, R¹², R¹³, R¹⁴, R¹⁵, and R¹⁶ are selected from hydrophilic groups of the formula L^{a}_{z}-R^{a}, wherein La is a hydrocarbon linker containing 1-12 carbon atoms, z is 0 or 1 (which indicates absence or presence of La, respectively), and R^{a} is independently selected from —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂, or R^{a} may be a group containing any of the foregoing hydrophilic groups. In some embodiments, any one or more of R¹¹-R¹⁶ contains an L^{a} and/or L^{c} linker that is or includes a polyethylene glycol (PEG) linker, wherein the PEG linker may include any suitable number of EG units, e.g., 1, 2, 5, 10, 12, 15, 20, 30, 40, or 50 EG units, or a number of units within a range therebetween (e.g., 1-50, 1-20, 1-12, 1-6, 2-50, 2-20, 2-12, or 2-6).

Moreover, in typical embodiments, M in Formula (3) is selected from any one of the following radionuclides: ¹⁷⁷Lu³⁺, ⁴³Sc³⁺, ⁴⁴Sc³⁺, ⁴⁷Sc³⁺, ⁶⁶Ga³⁺, ⁶⁷Ga³⁺, ⁶⁸Ga³⁺, ⁸⁶Y³⁺, ⁸⁸Y³⁺, ⁹⁰Y³⁺, ⁸⁹Zr⁴⁺, ¹¹¹In³⁺, ^{110m}In³⁺, ^{114m}In³⁺, ¹³⁵La³⁺, ¹³²La³⁺, ¹³⁴La³⁺, ¹³⁴Ce³⁺, ¹³⁷Ce³⁺, ¹⁵²Dy³⁺, ¹⁴⁹Tb³⁺, ¹⁵²Tb³⁺, ¹⁵⁵Tb³⁺, ¹⁶¹Tb³⁺, ²¹³Bi³⁺, ²¹²Bi³⁺, ²²⁷Th⁴⁺, ²²⁶Th⁴⁺, ²²⁵Ac³⁺, ²²⁶Ac³⁺, ²²³Ra²⁺, ²²⁴Ra²⁺, ¹³¹Ba²⁺, ²¹²Pb²⁺, ²¹²Pb⁴⁺, ²⁵⁵Fm³⁺, ²³⁰U⁶⁺, ²³⁰U⁴⁺, ²⁰¹Tl, ⁹⁰Nb, ¹⁴⁹Pm³⁺, ²³⁹Np⁴⁺, ²³⁹Np⁵⁺, ¹¹⁹Sb³⁺, ¹¹⁷Sb³⁺, ¹¹⁹Sb⁵⁺, ¹¹⁷Sb⁵⁺, ^{52g}Mn, ⁶⁴Cu, ⁴⁵Ti⁴⁺, ⁵⁵Co, and ^{58m}Co. Any sub-group of the foregoing radionuclides is also considered. For example, in some embodiments, M is selected from any one of the following radionuclides: ²²⁵Ac³⁺, ²²⁶Ac³⁺, ¹⁷⁷Lu³⁺, ¹¹¹In³⁺, ^{110m}In³⁺, ^{114m}In³⁺, ⁴³Sc³⁺, ⁴⁴Sc³⁺, and ⁴⁷Sc³⁺, or a sub-grouping thereof.

As noted above, L^{c} in Formula (3) may, in some embodiments, be a linking particle. The linking particle should be suitable for administration to living organisms, particularly humans. The particle may be a microparticle or nanoparticle. In embodiments, the particle is approximately or precisely spherical in shape and has a diameter of up to or less than 1 micron. The particle may have an organic or inorganic composition. Some organic compositions include polyesters (e.g., PLA, PGA, or PCL) and polyvinylalcohol (PVA).

Some inorganic compositions include silicon oxide, titanium dioxide, magnesium oxide, zinc oxide, and iron oxide. The diameter of the particle may be, for example, 5, 10, 20, 50, 100, 150, 200, 250, 300, 400, 500, 600, 700, 800, or 900 nm, or a size within a range bounded by any two of the foregoing values (e.g., 5-900 nm, 10-900 nm, 20-900 nm, 50-900 nm, 5-500 nm, 10-500 nm, 20-500 nm, 50-500 nm, 5-200 nm, 10-200 nm, 20-200 nm, 50-200 nm, 5-100 nm, 10-100 nm, 20-100 nm, 50-100 nm, 5-50 nm, 10-50 nm, 20-50 nm, 5-20 nm, 10-20 nm, or 5-10 nm).

In specific embodiments of Formula (3), the compound has the following structure:

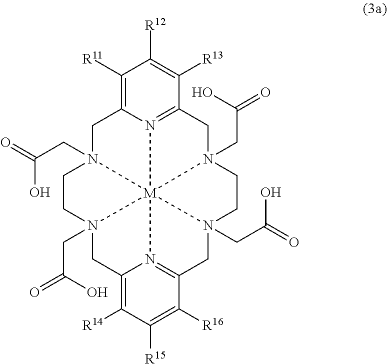

In Formula (3a), R¹¹, R¹², R¹³, R¹⁴, R¹⁵, and R¹⁶ groups are as defined and exemplified above under Formula (3). Any selection of the foregoing groups may be made in Formula (3a), and this selection may be further combined with any selection of M. In typical embodiments, M in Formula (3a) is selected from any one of the following radionuclides: ¹⁷⁷Lu³⁺, ⁴³Sc³⁺, ⁴⁴Sc³⁺, ⁴⁷Sc³⁺, ⁶⁶Ga³⁺, ⁶⁷Ga³⁺, ⁶⁸Ga³⁺, ⁸⁶Y³⁺, ⁸⁸Y³⁺, ⁹⁰Y³⁺, ⁸⁹Zr⁴⁺, ¹¹¹In³⁺, ^{110m}In³⁺, ^{114m}In³⁺, ¹³⁵La³⁺, ¹³²La³⁺, ¹³⁴La³⁺, ¹³⁴Ce³⁺, ¹³⁷Ce³⁺, ¹⁵²Dy³⁺, ¹⁴⁹Tb³⁺, ¹⁵²Tb³⁺, ¹¹⁵Tb³⁺, ¹⁶¹Tb³⁺, ²¹³Bi³⁺, ²¹²Bi³⁺, ²²⁷Th⁴⁺, ²²⁶Th⁴⁺, ²²⁵Ac³⁺, ²²⁶Ac³⁺, ²²³Ra²⁺, ²²⁴Ra²⁺, ¹³¹Ba²⁺, ²¹²Pb²⁺, ²¹²Pb⁴⁺, ²⁵⁵Fm³⁺, ²³⁰U⁶⁺, ²³⁰U⁴⁺, ²⁰¹Tl, ⁹⁰Nb, ¹⁴⁹Pm³⁺, ²³⁹Np⁴⁺, ²³⁹Np⁵⁺, ¹¹⁹Sb³⁺, ¹¹⁷Sb³⁺, ¹¹⁹Sb⁵⁺, ¹¹⁷Sb⁵⁺, ^{52g}Mn, ⁶⁴Cu, ⁴⁵Ti⁴⁺, ⁵⁵Co, and ^{58m}Co. Any sub-group of the foregoing radionuclides is also considered. For example, in some embodiments, M is selected from any one of the following radionuclides: ²²⁵Ac³⁺, ²²⁶Ac³⁺, ¹⁷⁷Lu³⁺, ¹¹¹In³⁺, ^{110m}In³⁺, ^{114m}In³⁺, ⁴³Sc³⁺, ⁴⁴Sc³⁺, and ⁴⁷Sc³⁺, or a sub-grouping thereof.

The macrocyclic chelators described herein can be made by means well known in the art, with some modifications, if needed. Detailed synthetic methods are also described in the Examples section. In typical embodiments, a py₂[18]aneN₆ precursor macrocycle can be reacted with a suitable functionalized electrophilic molecule to attach the L¹-R¹, L²-R², L³-R³, and L⁴-R⁴ groups, as shown in Formula (1), onto the four saturated nitrogen atoms in the py₂[18]aneN₆ precursor. For example, to synthesize PYTA, as shown in Formula (1b) complexed to M, the py₂[18]aneN₆ precursor may initially be reacted with four equivalents of tert-butyl bromoacetate followed by deprotection to remove the tert-butyloxycarbonyl (Boc) protecting group followed by acidification to yield the tetra-carboxy PYTA molecule or derivative thereof. Macrocyclic chelators containing one or more functional groups on the pyridine ring of the chelator (i.e., corresponding to R⁵, R⁶, R⁷, R⁸, R⁹, and/or R¹⁰) can be similarly synthesized, as above, but with an additional step of attaching a functional group on one or both pyridine rings. As further described in the Examples section, one or more protection-deprotection steps may be included to incorporate functional groups on the pyridine ring(s) and on the saturated nitrogen atoms. Complexation of the resulting macrocyclic chelator to a radionuclide can be done by means well known in the art along with modification of the conventional process, if needed, to bind certain radionuclides. The conjugation of a chelator complex to a cancer cell targeting group (e.g., monoclonal antibody) can be achieved using conventional conjugation methods, such as by reacting a reactive NCO or NCS group on the chelator with a cancer cell targeting group containing a primary amine or alcohol group.

To prepare a chelator compound radioisotope complex described herein, a chelator compound, such as any of those shown within the scope of Formulas (1), (2), or (3), are complexed with an appropriate radioisotope. Methods of preparing radionuclide complexes are well known in the art. For example, the radioisotope can be added to water and treated with an excess or at least an equimolar amount of the chelator compound. The chelator compound can be added as an aqueous solution or suspension, wherein aqueous solutions or suspensions include those containing binary solvent mixtures such as water-ethanol, water-DMSO, water-acetonitrile, and the like. Dilute acid or base can be added (where appropriate) to maintain a suitable pH. Heating at temperatures as high as 100° C. for periods of up to 24 hours or more may be employed to facilitate complexation, depending on the radioisotope, the chelator compound, and their concentrations.

In another aspect, the invention is directed to pharmaceutical compositions that contain any of the above-described radionuclide-containing complexes dispersed in a pharmaceutically acceptable vehicle (excipient). The composition can be dispersed in the pharmaceutically acceptable vehicle by either being mixed (e.g., in solid form with a solid carrier) or dissolved or emulsified in a liquid excipient. The pharmaceutical composition may or may not also be formulated together with one or more additional active ingredients or adjuvants that improve the overall efficacy of the pharmaceutical composition, particularly as relates to the treatment of cancer, infection by a microbe (e.g., a bacterium, virus, or fungus), or imaging of biological tissue.

The composition and vehicle (liquid or solid) may be formulated into pharmaceutical compositions and dosage forms according to methods well known in the art. The pharmaceutical compositions of the present invention may be specially formulated for administration in liquid or solid form. In some embodiments, the pharmaceutical formulation is formulated for oral administration (e.g., as tablets, capsules, powders, granules, pastes, solutions, suspensions, drenches, or syrups); parenteral administration (e.g., by subcutaneous, intramuscular or intravenous injection as provided by, for example, a sterile solution or suspension); topical application (e.g., as a cream, ointment, or spray); intravaginal or intrarectal administration (e.g., as a pessary, cream or foam); sublingual or buccal administration; ocular administration; transdermal administration; or nasal administration. In some embodiments, the pharmaceutical composition is a liquid formulation designed for administration by injection.

The pharmaceutical composition can be made suitable for use as a medicament for human or animal use. The pharmaceutical composition may be, for example, an injectable formulation, a liquid, cream or lotion for topical application, an aerosol, a powder, granules, tablets, suppositories, or capsules, such as, for example, enteric coated capsules, or the like. The pharmaceutical composition may also be delivered as part of a lipid formulation, such as, for example, an emulsion or a liposome preparation. The pharmaceutical compositions are preferably sterile, non-pyrogenic and isotonic preparations, optionally with one or more of the pharmaceutically acceptable additives listed below. Pharmaceutical compositions are preferably stable compositions which may contain one or more of the following: a stabilizer, a surfactant, preferably a nonionic surfactant, and optionally a salt and/or a buffering agent. The pharmaceutical composition may be in the form of an aqueous solution, or may be in a lyophilized form. The stabilizer may, for example, be an amino acid, such as for instance, glycine; or an oligosaccharide, such as for example, sucrose, tetralose, lactose, or a dextran. Alternatively, the stabilizer may be a sugar alcohol, such as for instance, mannitol; or a combination thereof. Preferably, the stabilizer or combination of stabilizers constitutes from about 0.1% to about 10% weight for weight of the composition. The surfactant may be a nonionic surfactant, such as a polysorbate. Some examples of suitable surfactants include Tween20, Tween80; a polyethylene glycol or a polyoxyethylene polyoxypropylene glycol, such as Pluronic F-68 at from about 0.001% (w/v) to about 10% (w/v). The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal. Preferably, the salt or buffering agent is present at a roughly isotonic concentration of about 150 mM to about 300 mM.

The pharmaceutical composition may additionally contain one or more conventional additives. Some examples of such additives include a solubilizer, such as, for example, glycerol; an antioxidant, such as, for example, benzalkonium chloride (a mixture of quaternary ammonium compounds, known as “quats”), benzyl alcohol, chloretone or chlorobutanol; an anesthetic agent, such as, for example, a morphine derivative; or an isotonic agent, such as described above. As a further precaution against oxidation or other spoilage, the pharmaceutical compositions may be stored under nitrogen or argon gas in vials sealed with impermeable stoppers.

The phrase “pharmaceutically acceptable” refers herein to those compounds, materials, compositions (e.g., acids or bases), and/or dosage forms which are, within the scope of sound medical judgment, suitable for administration to a subject. The phrase “pharmaceutically acceptable vehicle,” as used herein, refers to a pharmaceutically acceptable liquid or solid filler, diluent, carrier, manufacturing aid (e.g., lubricant, talc magnesium, calcium or zinc stearate, or stearic acid), solvent, or encapsulating material, that serves to carry the therapeutic composition for administration to the subject. Each vehicle (excipient or diluent) should be “acceptable” in the sense of being compatible with the other ingredients of the formulation and physiologically safe to the subject. Any of the vehicles/excipients known in the art can be suitable herein depending on the mode of administration.

Some examples of materials that can serve as pharmaceutically acceptable vehicles include water; aqueous and non-aqueous solutions; isotonic saline; pH buffering agents; anti-oxidants; bacteriostats; sugars (e.g., lactose, glucose, sucrose, and oligosaccharides, such as sucrose, trehalose, lactose, or dextran); solutes that render the formulation isotonic with the blood of the intended recipient; and aqueous and non-aqueous sterile suspensions that can include suspending agents, solubilizers, thickening agents, stabilizers, and preservatives. Other excipients, more typically used in solid dosage forms, may also be included, e.g., starches (e.g., corn and potato starch); cellulose and its derivatives (e.g., sodium carboxymethyl cellulose, ethyl cellulose and cellulose acetate); gelatin; talc; waxes; oils (e.g., peanut oil, cottonseed oil, safflower oil, sesame oil, olive oil, corn oil and soybean oil); glycols (e.g., ethylene glycol, propylene glycol, and polyethylene glycol); polyols (e.g., glycerin, sorbitol, and mannitol); esters (e.g., ethyl oleate and ethyl laurate); agar; and other non-toxic compatible substances employed in pharmaceutical formulations. If desired, certain sweetening and/or flavoring and/or coloring agents may be added. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995). The formulations can be presented in unit-dose or multi-dose sealed containers, such as ampules and vials, and can be stored in a freeze-dried (lyophilized) condition requiring only the addition of the sterile liquid carrier, such as, for example, water, for injections, immediately prior to use. Extemporaneous injection solutions and suspensions can be prepared from sterile powders, granules, and tablets of the kind previously described.

In another aspect, the present disclosure is directed to a method of treating cancer in a subject. In the method, a pharmaceutically effective amount of a macrocycle-radionuclide complex according to Formula (3) or sub-formula thereof is administered to a subject afflicted with cancer. The method of treating cancer may include selectively targeting cancerous tissue by including a cancer targeting moiety, such as any of those described above, attached to the radionuclide-containing composition. In some embodiments, the radionuclide (M) used in treating the cancer is selected from ¹⁷⁷Lu³⁺, ⁴⁷Sc³⁺, ⁹⁰Y³⁺, ¹³⁵La³⁺, ¹⁵²Dy³⁺, ¹⁶¹Tb³⁺, ¹⁸⁸Re⁺¹, ¹⁸⁶Re⁺¹, ²¹³Bi³⁺, ²¹²Bi³⁺, ²¹¹At, ²²⁷Th⁴⁺, ²²⁶Th⁴⁺, ²²⁵Ac³⁺, ²²³Ra²⁺, ²²⁴Ra²⁺, ²¹²Pb²⁺, ²¹²Pb⁴⁺, ²⁵⁵Fm³⁺, ²³⁰U⁶⁺, ²³⁰U⁴⁺, ²⁰¹Tl, ¹⁴⁹Pm³⁺, ²³⁹Np⁴⁺, ²³⁹Np⁵⁺, ¹¹⁹Sb³⁺, ¹¹⁹Sb⁵⁺, ⁶⁷Cu, ¹⁵³Sm, and ¹⁶⁶Ho

Since a cancer cell targeting agent can be selected to target any of a wide variety of cancers, the cancer considered herein for treatment is not limited. The cancer can be essentially any type of cancer. For example, antibodies or peptide vectors can be produced to target any of a wide variety of cancers. The targeting compositions described herein are typically administered by injection into the bloodstream, but other modes of administration, such as oral or topical administration, are also considered. In some embodiments, the targeting composition may be administered locally, at the site where the target cells are present, i.e., in a specific tissue, organ, or fluid (e.g., blood, cerebrospinal fluid, etc.). Any cancer that can be targeted through the bloodstream is of particular consideration herein. The cancer may be, for example, prostate cancer, ovarian cancer, breast cancer, leukemia, colorectal cancer, lymphoma, lung cancer, stomach cancer, uterine cancer, pancreatic cancer, cervical cancer, liver cancer, kidney cancer, skin cancer, neuroblastoma, and brain cancer. The cancer can also include the presence of one or more carcinomas, sarcomas, lymphomas, blastomas, or teratomas (germ cell tumors). In some embodiments, the cancer is a triple negative breast cancer. The method of treating cancer may additionally (i.e., sequentially or simultaneously) include imaging of cancerous tissue that has accumulated the radionuclide-containing composition. The imaging can be achieved by any of the nuclear medicine imaging techniques known in the art.

A “pharmaceutically effective amount” refers to the amount required to produce a desired effect in treating the cancer. One non-limiting example of an effective amount includes amounts or dosages that yield acceptable toxicity and bioavailability levels for therapeutic (pharmaceutical) use including, but not limited to, the treatment of e.g., prostate cancer, breast cancer, or bladder cancer. Another example of an effective amount includes amounts or dosages that are capable of reducing symptoms associated with e.g., prostate cancer, breast cancer, or bladder cancer, such as, for example, reduction in proliferation and/or metastasis of prostate cancer, breast cancer, or bladder cancer. Yet, another example of an effective amount includes amounts or dosages that are suitable for imaging, such as imaging of cancel, such as e.g., prostate cancer, breast cancer, or bladder cancer. The effective amount may be from about 0.01 μg to about 1 mg of the compound per gram of the composition, and preferably from about 0.1 μg to about 500 μg of the compound per gram of the composition. As used herein, a “subject” or “patient” is a mammal, such as a cat, dog, rodent or primate. Typically, the subject is a human, and, preferably, a human suffering from or suspected of suffering from cancer, such as non-small cell lung cancer, bladder cancer, or colon cancer (such as colon adenocarcinoma). The term “subject” and “patient” can be used interchangeably.

In the method of treating cancer, an effective amount of the composition, typically in a pharmaceutical composition, may be administered to a human or an animal in need thereof by any of a number of well-known methods. For example, the composition may be administered systemically or locally, such as by injection. The systemic or local administration of the composition may be by intravenous, subcutaneous, intraperitoneal, intramuscular, intrathecal, or oral administration. An effective amount of a pharmaceutical composition of the invention is any amount that is effective to achieve its purpose. The effective amount, usually expressed in mg/kg can be determined by routine methods during pre-clinical and clinical trials by those of skill in the art. Dosing is dependent on the severity and responsiveness of the disease or condition being treated, with the course of treatment possibly lasting from several days to several months, or until a cure is effected or a diminution of the disease state is achieved. Optimal dosing schedules can be calculated from measurements of drug accumulation in the body of the patient. The administering physician can determine optimum dosages, dosing methodologies, and repetition rates. The dose administered to the subject should be sufficient to result in a therapeutic response in the subject over a reasonable time frame to treat an illness or condition, and/or for diagnostic imaging of the desired tissue or organ. The dose can be determined by the potency of the particular compositions employed and the condition of the subject as well as the body weight of the animal (e.g., human) to be treated. The size of the dose can also be determined by the nature and extent of any adverse side effects that may accompany the administration of a particular composition. A typical dosage for internal administration is 0.01 to 100 mg/kg of body weight per day, such as 0.01 to 35 mg/kg of body weight per day or 0.05 to 5 mg/kg of body weight per day. A typical concentration of the composition in pharmaceutical compositions for topical administration is 0.05 to 15% (by weight), preferably 0.02 to 5%, and more preferably 0.1 to 3%.

In different embodiments, depending on these and other factors, a suitable dosage of the radionuclide-containing composition may be precisely, at least, or no more than, for example, 1 mg, 10 mg, 50 mg, 100 mg, 200 mg, 300 mg, 400 mg, 500 mg, 600 mg, 700 mg, 800 mg, 900 mg, 1000 mg, 1200 mg, or 1500 mg, per 50 kg, 60 kg, or 70 kg adult, or a dosage within a range bounded by any of the foregoing exemplary dosages. Depending on these and other factors, the composition is administered in the indicated dosage or within a range thereof by any suitable schedule, e.g., once, twice, or three times a day for a total treatment time of one, two, three, four, or five days, and up to, for example, one, two, three, or four weeks or months. The indicated dosage may alternatively be administered every two or three days, or per week. Alternatively, or in addition, the composition can be administered until a desired change in the disorder or condition is realized, or when a preventative effect is believed to be provided. The targeting composition may or may not be co-administered with one or more anti-cancer drugs, such as cis-platin, 5-fluorouracil, or bleomycin, which may be given in the same or different amount, such as any of the amounts provided above.

The pharmaceutical composition may be packaged in unit dosage form. The unit dosage form is effective in imaging or for treating cancer, such as non-small cell lung cancer, bladder cancer, or colon cancer (such as colon adenocarcinoma). Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to powders, tablets, pills, capsules, lozenges, suppositories, patches, nasal sprays, injectables, implantable sustained-release formulations, adherent films, topical varnishes, lipid complexes, and the like.

The term “treating,” as used herein, refers to any amelioration of the cancer, such as a reduction or statis in a tumor size or a marker or indicator of the cancer. Various assays and model systems can be readily employed to determine the therapeutic effectiveness of the treatment. In some embodiments, test subjects will exhibit a 10%, 20%, 30%, 50% or greater reduction, up to a 75-90%, or 95% or greater, reduction, in one or more symptom(s) caused by or associated with the cancer in the subject, compared to placebo-treated or other suitable control subjects.

In yet another aspect, the present disclosure is directed to a method of imaging biological (i.e., bodily) tissue by administering a radionuclide-containing composition within the scope of Formula (3). Depending on the radionuclide selected, the imaging method may be performed in concert with the treatment of cancer described above, or the imaging method may be performed solely for the purpose of imaging cancerous tissue. In the imaging method, a pharmaceutically effective amount (i.e., sufficient for imaging) of a macrocycle-radionuclide complex according to Formula (3) or sub-formula thereof is administered to a subject, followed by imaging of biological tissue in the subject. The subject is typically afflicted with a cancer, and the complex typically functions to image cancerous tissue in the subject. The imaging method may include selectively targeting cancerous tissue by including a cancer targeting moiety, such as any of those described above, attached to the radionuclide-containing composition. In some embodiments, the radionuclide (M) used in imaging the bodily tissue is selected from ⁴³Sc³⁺, ⁴⁴Sc³⁺, ⁶⁶Ga³⁺, ⁶⁷Ga³⁺, ⁶⁸Ga³⁺, ⁸⁶Y³⁺, ⁸⁸Y³⁺, ⁸⁹Zr⁴⁺, ^{99m}Tc⁺¹, ¹¹¹In³⁺, ^{110m}In³⁺, ¹³²La³⁺, ¹³⁴La³⁺, ¹³⁴Ce³⁺, ¹³⁷Ce³⁺, ¹⁵²Dy³⁺, ¹⁴⁹Tb³⁺, ¹⁵²Tb³⁺, ¹⁵⁵Tb³⁺, ²²⁶Ac³⁺, ¹³¹Ba²⁺, ^{133m}Ba²⁺, ⁹⁰Nb, ¹¹⁷Sb³⁺, ¹¹⁷Sb⁵⁺, ^{52g}Mn, ⁶⁴Cu, and ⁴⁵Ti⁴⁺.

In particular embodiments, the method entails administering to a subject a radionuclide-containing composition described above in an amount effective to provide an image and exposing the host to an energy source, whereupon a diagnostic image of the host is obtained. The diagnostic image can be, for example, a magnetic resonance image (MRI), fluorescence image (FI), x-ray contrast image, transmission electron microscopy image, positron emission tomography (PET) image, single photon emission computed spectroscopy (SPECT), or other type of diagnostic image.

Examples have been set forth below for the purpose of illustration and to describe certain specific embodiments of the invention. However, the scope of this invention is not to be in any way limited by the examples set forth herein.

### EXAMPLES

### Synthesis of PYTA Macrocyclic Chelator

PYTA, as shown in Formula (1b), was prepared according to a known method starting from py2[18]aneN₆·0.2HCl·3.8HBr·0.5H2O, with slight modifications (L. Valencia et al., Inorg. Chem., 2002, 41, 5300-5312). Specifically, 4.3 equiv of ethyl bromoacetate and 12 equiv of Na₂CO₃ were used in the alkylation reaction. Following extraction, the crude material was heated for 2 days (unoptimized) in 6 M HCl at 90° C. to hydrolyze the ester groups. Purification of the product was achieved by semi-preparative reverse-phase C₁₈ high-performance liquid chromatography (HPLC) using an aqueous column (100 Å, 5 m, 250 mm×21.2 mm) and a MeOH/H₂O mobile phase containing 0.1% trifluoroacetic acid. PYTA·3.8 CF₃COOH was isolated as a white powder (1.347 g, 61% yield). The number of TFA counteranions per ligand varied slightly from batch to batch and is noted for each experiment. The purity and composition of macropa and PYTA were verified to be >99% by reverse-phase C₁₈ analytical HPLC (100 Å, 5 m, 250 mm×4.6 mm), elemental analysis, and ¹H and ¹³C{1H}nuclear magnetic resonance (NMR) spectroscopy (400 MHz spectrometer equipped with a BBO probe).

### Synthesis of In-PYTA Complex

PYTA 2.5 CF₃COOH (21.0 mg; 0.025 mmol), triethylamine (10.7 μL; 0.077 mmol), and EtOH (5 mL) were combined in a vial. The resulting solution was heated to 60° C. Then, a solution of In(ClO₄)₃·8H₂O (13.8 mg; 0.025 mmol) in EtOH (3 mL) was added dropwise. A white precipitate formed immediately. The suspension was stirred at 60° C. for 1 h and then concentrated to ~1 mL. An aliquot of H₂O (3 mL) was added to the suspension, and it was heated at 70° C. until dissolution of the precipitate was complete (~1 h). Crystals suitable for single-crystal X-ray diffraction were obtained from slow evaporation of this solution after several days and were determined to have the composition [In(H₂PYTA)]ClO₄.

### Synthesis of Sc-PYTA Complex

PYTA·3.8 CF₃COOH (22.4 mg; 0.023 mmol), triethylamine (10.7 μL; 0.077 mmol), and EtOH (3 mL) were combined in a vial. To this solution was added Sc(ClO₄)₃·6H₂O (50 wt % in H₂O; 19.7 mg; 0.022 mmol) in EtOH (3 mL). White precipitate formed immediately. The precipitate was isolated via centrifugation, washed with EtOH (6 mL), and then dissolved in H₂O (5 mL) with gentle heating (40° C.). The aqueous solution was then concentrated at 60° C. to ~2 mL. Crystals suitable for single-crystal X-ray diffraction were obtained from slow evaporation of this solution after 1 week and were determined to have the composition [Sc(HPYTA)]·3H₂O.

### Synthesis of Cu-PYTA Complex

PYTA·3.8 CF₃COOH (20.1 mg; 0.021 mmol), triethylamine (10.7 μL; 0.077 mmol) and EtOH (3 mL) were combined in a vial. The solution was heated at 70° C. for 15 min. To this solution was added Cu(ClO₄)₂·6H₂O (11.8 mg; 0.032 mmol) in EtOH (3 mL). The reaction solution immediately turned green. The reaction volume was reduced to ~3 mL. Crystals suitable for single-crystal X-ray diffraction were obtained from slow evaporation after ~2 weeks and were determined to have the composition [Cu(H₂PYTA)]2H₂O.

### Radionuclides

### Sourcing and Solution Preparation

Actinium-225 (²²⁵Ac) was obtained from a ²²⁹Th generator and received as a dried-down nitrate salt with a specific activity of 5.80×10⁴ Ci/g (carrier free) and a radionuclidic purity of 99.99%. Upon receipt, it was reconstituted in 10 mM HCl (ultra-trace elemental analysis grade H₂O) to yield a stock solution of approximately 74 kBq ²²⁵Ac/μL (2 μCi ²²⁵Ac/μL). From this stock solution, working solutions of 0.93-1.11 kBq/μL (0.025-0.03 μCi/μL) or 3.7 kBq/μL (0.1 μCi/μL) were prepared prior to radiolabeling and serum stability experiments, respectively, by further dilution with 10 mM HCl.

Lutetium-177 (¹⁷⁷Lu) was received as a chloride solution (0.05 M HCl) with a specific activity≥740 GBq/mg (≥20 Ci/mg) and a radionuclidic purity of ≥99%. From this stock solution, working solutions of 2.2-3.7 kBq/μL (0.06-0.1 μCi/μL) or 14.8 kBq/μL (0.4 μCi/μL) were prepared prior to radiolabeling and serum stability experiments, respectively, by further dilution with 10 mM HCl.

Indium-111 (¹¹¹In) was received as a chloride solution (0.05 M HCl) with a specific activity≥50 Ci/mg, an activity concentration of approximately 1,000 mCi/mL, and a radionuclidic purity of >99.9%. From this stock solution, working solutions of 7.4-11.1 kBq/μL (0.2-0.3 μCi/μL) or 18.5-29.6 kBq/μL (0.5-0.8 μCi/μL) were prepared prior to radiolabeling and serum stability experiments, respectively, by further dilution with 10 mM HCl.

Scandium-44 (⁴⁴Sc) was eluted in 0.05 M HCl from a ⁴⁴Ti/⁴⁴Sc generator, yielding a stock solution of 1.8 (±0.09) mCi in 3.6 mL (0.5 μCi/μL, 18.5 kBq/μL). The pH of the stock solution was adjusted to approximately 4.5 through addition of 5 M NaOH (25 μL) and NH₄OAc (0.25 M, pH 4.05). This solution was used for the labeling experiments below.

### Gamma Spectroscopy

The radionuclidic purity and activity of ²²⁵Ac, ¹⁷⁷Lu, and ¹¹¹In solutions were verified by gamma spectroscopy using a gamma analyst integrated gamma spectrometer that includes a high-purity germanium (HPGe) detector (model GC-GA1), U-type cryostat, and automatic sample changer. The detector energy and efficiency were calibrated using a mixed gamma point source containing ⁵⁷Co, ⁶⁰Co, ⁸⁸Y ¹⁰⁹Cd, ¹¹³Sn, ¹³⁷Cs, ¹³⁹Ce, ²⁰³Hg, and ²⁴¹Am, traceable to the National Institute of Standards and Technology (NIST). Samples to be counted were prepared by adding 10 μL of working solution into 12×75 mm polypropylene test tubes. Counting dead time was maintained below 5% for all measurements. Data was analyzed using commercial software. Spectral acquisitions of ⁴⁴Sc were made for 1 h on a commercial system. Samples were placed 5 cm offset from the aluminum endcap, enclosed in a 10 cm lead shield.

### Radiolabeling and Serum Stability Studies

### General

The structures of PYTA, macropa, and DOTA are provided as follows:

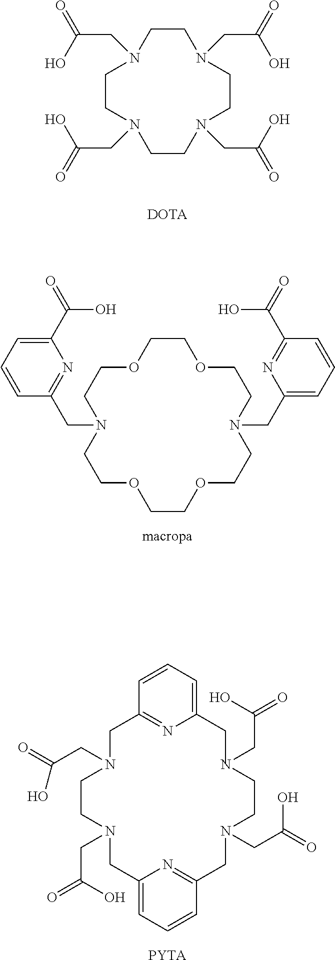

Stock solutions of PYTA, macropa, and DOTA were prepared in Milli-Q H₂O. The concentration of each solution was verified by potentiometric titration. Subsequently, the stocks were serially diluted with H₂O to afford a series of ligand solutions ranging from 1.5×10⁻⁷ to 1.5×10⁻² M. NH₄OAc buffer (0.5 M, pH 6.0) was prepared using solid NH₄OAc (99.999% trace metals basis) and glacial HOAc (≥99%), and was rendered metal-free by contact with Chelex® (5 g/L) overnight via the batch method. It was subsequently filtered through a polypropylene column equipped with a 20 μm polyethylene frit and stored in an acid-washed plastic bottle. Human serum from male AB plasma (sterile-filtered) was obtained from a commercial source.

The following plate/mobile phase systems were used for radio thin-layer chromatography (radio-TLC):

TLC System A: Mobile phase: 50 mM ethylenediaminetetraacetic acid (EDTA) at pH 6.0, prepared by dissolving a suspension of H₄EDTA (99.995% trace metals basis) in Milli-Q H₂O with NaOH. TLC plate: instant thin layer chromatography-silica gel (iTLC-SG) plates.

TLC System B: Mobile phase: 0.4 M sodium citrate with 10% MeOH, prepared by mixing sodium citrate buffer (0.4 M, pH 6) with MeOH (9:1 v/v). TLC plate: aluminum-backed silica TLC plates (alumTLC-SG, silica gel 60, F254).

TLC System C: Mobile phase: 10 mM ammonium acetate:MeOH (9:1 v/v). TLC plate: aluminum-backed reverse-phase TLC plates (alumTLC-RP, silica gel 60, RP-18, F254).

TLC System D: Mobile phase: 50 mM EDTA at pH 5.5. TLC plate: instant thin layer chromatography-silica gel (iTLC-SG) plates.

TLC System E: Mobile phase: saline. TLC plate: instant thin layer chromatography-silica gel (iTLC-SG) plates.

TLC System F: Mobile phase: 0.4 M sodium citrate with 10% MeOH, prepared by mixing sodium citrate buffer (0.4 M, pH 6) with MeOH (9:1 v/v). TLC plate: aluminum-backed reverse-phase TLC plates (alumTLC-RP, silica gel 60, RP-18, F254).

### Concentration-Dependent Radiolabeling

Radiolabeling experiments with ²²⁵Ac, ¹⁷⁷Lu, ¹¹¹In, and ⁴⁴Sc were performed in triplicate at each chelator concentration by addition of chelator (10 μL) and radionuclide working solution (10 μL; 9.3-11.1 kBq 225Ac, 22-37 kBq 177Lu, 74-111 kBq 111In, or 185 kBq 44Sc) to polypropylene screw-capped tubes containing pH 6 NH₄OAc buffer (130 μL), giving a final chelator concentration of 10⁻⁸-10⁻³ M. Control samples were also prepared in which chelator was substituted with H₂O. The samples were shaken to ensure complete mixing and then incubated at room temperature for 5 min. After removing an aliquot for radio-TLC, the samples were rotated end-over-end in an incubator at 25° C. until the next time point (60 min). Radiolabeling of DOTA at 80° C. was also performed for comparison. The pH of representative samples was confirmed to be 6 by spotting an aliquot (1 μL) onto a pH indicator strip. Additionally, radiolabeling experiments with 1.665 MBq of ⁴⁴Sc were executed for PYTA in triplicate in an analogous manner, except the final reaction volume was 100 μL, the final pH was approximately 4.5, and the mixing time was 30 min.

The radiolabeling progress was analyzed via radio-TLC by spotting an aliquot (5 μL) of reaction solution onto iTLC-SG, alumTLC-SG, or alumTLC-RP plates and developing them using the mobile phases described above. For ²²⁵Ac radio-TLC, strips were analyzed approximately 24 h after their development to allow sufficient time for radioactive equilibrium to be reached between ²²⁵Ac and its decay chain. ¹⁷⁷Lu, ¹¹¹In, and ⁴⁴Sc radio-TLC strips were analyzed immediately. TLC imaging was performed using a commercial scanner system equipped with P-10 gas and commercial imaging software. Radiochemical yields (RCYs) were calculated by dividing the counts associated with complexed radionuclide by the total counts integrated along the length of the TLC plate.

### Serum Challenges

Chelators were radiolabeled at desired concentrations for 60 min at 25° C. (PYTA, macropa) or 80° C. (DOTA) with ²²⁵Ac (37 kBq), ¹⁷⁷Lu (148 kBq), ¹¹¹In (185-296 kBq), or ⁴⁴Sc (740 kBq, PYTA only) in pH 6.0 HOAc—NH₄OAc buffer (150 μL final volume). Each concentration of chelator was selected based on the lowest concentration that gave quantitative or nearly quantitative RCYs in radiolabeling experiments, multiplied by a factor of 4 to account for subsequent dilution by serum. RCYs of >97% were confirmed by radio-TLC for all radiolabeling reactions except for the [¹⁷⁷Lu]Lu-macropa reaction, which had a RCY of 82%. Next, an aliquot of human serum (450 μL) was added to each sample, yielding a final reaction volume of 600 μL and final chelator concentrations ranging from 1×10⁻⁵ M to 1×10⁻³ M. Control samples were also prepared in which chelator was substituted with H₂O and diluted with 450 μL of serum. The samples were rotated end-over-end at 37° C. and sampled at predetermined time points. The percentage of intact complex was determined by radio-TLC, as described above. The final pH of representative samples was determined to be 7 by spotting an aliquot (1 μL) of reaction solution onto a pH indicator strip.

### PET Imaging and Ex Vivo Biodistribution

### General

All radioactive material handling and animal experimentation were conducted in compliance with institutional regulations and approved by Environmental Health and Safety Radioactive Materials protocol #1169-01 and Institutional Animal Care and Use Committee protocol #22-0023. Female Swiss Webster mice (6-8 weeks) were purchased for imaging and biodistribution studies. A total of 16 mice were used for the biodistribution study of the ²²⁵Ac-PYTA complex and 12 mice for the [⁴⁴Sc]Sc-PYTA complex, with n=4 per time point. Additionally, 4 mice were used for the [⁴⁴Sc]Sc-PYTA PET imaging study.

²²⁵Ac Studies

PYTA (1×10⁻³ M) was radiolabeled with ²²⁵Ac (185 kBq, 5 μCi) at room temperature in NH₄OAc buffer (0.25 M, pH 6) containing gentisic acid (1 mM) and ascorbic acid (1 mM). The final volume of the reaction solution was 500 μL. After 60 min, the reaction was spotted onto an alumTLC-SG strip and developed using TLC system B, which confirmed >98% RCY. The reaction was allowed to proceed for 48 h and then re-sampled, and the RCY was again confirmed to be >98%. At this time, the reaction solution was diluted 10-fold with saline for intravenous administration. Mice were anesthetized by isoflurane in air (2%, 2 L/min), and approximately 100 μL (~3.7 kBq, 100 nCi) of ²²⁵Ac complex was administered intravenously to mice by retro-orbital injection. Each group was sacrificed after 15 min, 1 h, 24 h, or 48 h post-injection. Selected tissues and biological fluids were collected, weighed, and gamma counted at equilibrium (>4 h). Counts were decay corrected from the time of injection and then converted to the percentage of injected activity (% IA) per gram of weighed tissue (% IA/g).

### **Sc Studies

PYTA (5.7 mM, 15 L) was radiolabeled with ⁴⁴Sc (33.5 MBq, 906 μCi) at room temperature in NH₄OAc buffer (0.25 M, pH 6). The final volume of the reaction solution was 1.5 mL. After 40 min, the reaction was spotted onto an alumTLC-RP strip and developed using TLC system C, which confirmed >98% RCY. After the reaction, a Sep-Pak C18 cartridge was used to purify the final compound. The labeled compound was eluted using EtOH and diluted with saline for intravenous administration. Radiolabeled solutions were prepared fresh for PET imaging and biodistribution studies, which were carried out on different days. Mice were anesthetized by isoflurane in air (2%, 2 L/min), and approximately 100 μL (~5.18 MBq, 140 μCi) of ⁴⁴Sc complex was administered to mice by intravenous tail vein catheter for dynamic imaging. Animals were imaged using the microPET R4 and data was corrected for detector non-uniformity, dead time, random coincidences and physical decay. An energy window of 350-650 keV and coincidence-timing window of 6 ns was used. Activity concentration calibrated data was reconstructed and visualized using commercial software. Animals for biodistribution were administered activity intravenously by the retroorbital sinus (~2.775 MBq, 75 μCi in 100 μL) and were sacrificed after 15 min, 1 h, or 4 h post-injection (n=4 per time point). Selected tissues and biological fluids were collected, weighed, and gamma counted. Counts were decay corrected from the time of injection and then converted to the percentage of injected activity (% IA) per gram of weighed tissue (% IA/g) determined by automated microbalance.

As a first assessment of the suitability of PYTA for the chelation of ²²⁵Ac, ¹⁷⁷Lu, ¹¹¹In, and ⁴⁴Sc, its solution-state chemistry with La³⁺, Lu³⁺, In³⁺, and Sc³⁺ was probed by ¹H nuclear magnetic resonance (NMR) spectroscopy in D₂O. Ac³⁺ was substituted with La³⁺ for this study because Ac³⁺ does not possess any stable isotopes, rendering the obtention of NMR spectra challenging. La³⁺ is often used as a surrogate for Ac³⁺ because of its similar chemical properties and only slightly smaller ionic radius (1.160 Å for 8-coordinate La³⁺, 1.220 Å for 9-coordinate Ac³⁺). The present density functional theory calculations and ab initio molecular dynamics simulations reveal similarities between La³⁺ and Ac³⁺ coordination by PYTA, supporting the notion that the La³⁺ complex approximates the Ac³⁺ complex in solution. NMR samples were prepared at pH~7 using equimolar amounts of chelator and metal ion, and their spectra were acquired after 1 h at room temperature, except for the La³⁺-PYTA sample, which required further heating at 60° C. (unoptimized) to drive the complexation reaction to completion. High-resolution mass spectrometry confirmed complex formation for all samples.

The NMR spectra obtained for La³⁺ and Lu³⁺ were similar to those reported previously. Specifically, complexation of La³⁺ by PYTA gave rise to 8 sharp signals, indicating the formation of a D₂-symmetric complex in which all pendent arms are bound to the metal center in a rigid 10-coordinate configuration. By contrast, the Lu³⁺ spectrum was consistent with an asymmetric 9-coordinate complex. This configuration was evident from the presence of 2 triplets and 2 doublets for the 6 pyridyl protons of the chelator, which indicates that the pyridyl groups experience different magnetic environments. Additionally, a singlet was observed at 4.22 ppm, which can be assigned to the methylene protons of one uncoordinated acetate arm.

The spectrum of the Sc-PYTA complex matched closely to that of the Lu-PYTA complex, indicating a shared 9-coordinate structural conformation. By contrast, the spectrum of the In-PYTA complex was less straightforward to interpret. Broadening of the aliphatic resonances precluded any meaningful analysis of this region. However, two sets of signals can be discerned in the aromatic region in approximately a 65/35 ratio, one set belonging to a symmetric species (major) and the other to an asymmetric species (minor). Upon heating the sample to 90° C., the two sets of aromatic signals were observed to coalesce. These findings are consistent with the existence of two configurational isomers in solution undergoing slow exchange on the NMR timescale. Another notable feature of the In-PYTA spectrum is that the aromatic resonances are shifted considerably downfield in comparison to those of the other complexes. This shift may signify that the pyridyl groups of PYTA are positioned closer to the electron-withdrawing metal center when chelating In³⁺ versus La³⁺, Lu³⁺, and Sc³⁺. Collectively, the differences observed in the spectra of these complexes in aqueous solution point towards the flexibility of PYTA to adopt the configurations necessary to satisfy the coordination preferences of a range of metal ions.

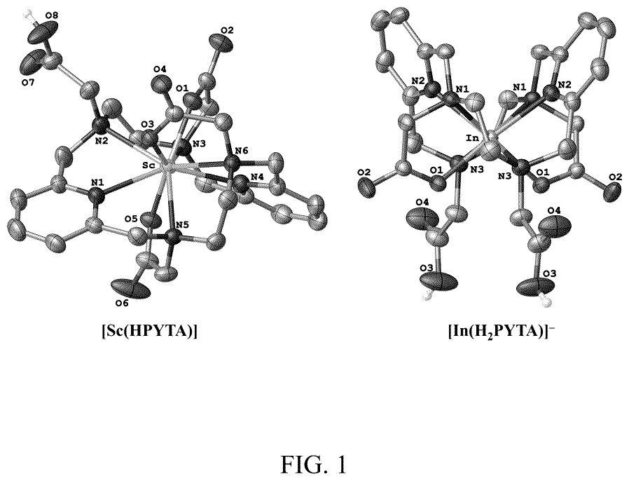

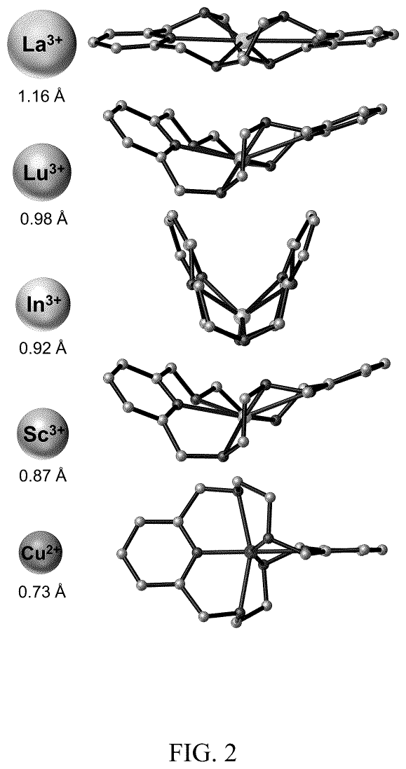

To further determine the structural changes that PYTA undergoes upon binding these metals, single-crystal structures of its Sc³⁺ and In³⁺ complexes were obtained, as shown in FIG. 1, and these were compared to the published crystal structures of its La³⁺ and Lu³⁺ complexes, [L^{a}(H₂PYTA)](NO₃)·3H₂O and [Lu(HPYTA)]·3H₂O (L. Valencia et al., Inorg. Chem., 2002, 41, 5300-5312). Select interatomic distances and angles are presented in Table 1 below. The Sc³⁺ complex crystallized in the C2/c space group as [Sc(HPYTA)]·3H₂O (FIG. 1), with a conformation that is isostructural to that of the Lu³⁺ complex. Specifically, 3 of the 4 pendant arms are deprotonated and engaged in binding interactions with the metal center, giving rise to a 9-coordinate complex that is consistent with our NMR analysis. Additionally, the pyridine rings of the core macrocycle are twisted away from one another and folded inward towards the metal center (FIG. 2), giving rise to a N_{py}−M−N_{py} angle of 146.04(5)° (Table 1). This “twist-fold” conformation is typical for PYTA when complexing smaller lanthanide ions, whereas the macrocycle adopts a more planar structure when binding larger ions like La³⁺ (FIG. 2, N_{py}−M−N_{py}=178.40(7)°).

TABLE 1
Comparison of average interatomic distances (Å) and angles (°) observed in
the X-ray crystal structures of La³⁺, Lu³⁺, In³⁺, and Sc³⁺ complexes of PYTA^{a}

| | La^{b} | Lu^{b} | In^{c} | Sc^{c} |
|---|---|---|---|---|
| Ionic | 1.160 | 0.977 | 0.92 | 0.870 |
| Radius^{d} | | | | |
| Space Group | P2₁/n | C2/c | R3̅c | C2/c |
| M—N_{py} | 2.637(1) | 2.508(2) | 2.3401(10) | 2.463(4) |
| M—Nₐₘᵢₙₑ | 2.702(10) | 2.594(27) | 2.523(89) | 2.578(49) |
| M—O_{carb}^{e} | 2.604(69) | 2.250(5) | 2.1328(10) | 2.137(5) |
| N_{py}—M—N_{py} | 178.40(7) | 146.45(7) | 108.58(5) | 146.04(5) |

^{a}Values in parentheses are one standard deviation (of the last significant figures) from the average of crystallographically independent values. In instances where there is only one crystallographically independent value, the standard uncertainty of the refined value is reported.
^{b}From L. Valencia et al., Inorg. Chem., 2002, 41, 5300-5312.
^{c}This work.
^{d}Eight-coordinate ionic radii from R. D. Shannon, Acta Crystallogr., Sect. A: Cryst. Phys., Diffr., Theor. Gen. Crystallogr., 1976, 32, 751-767.
^{e}Averages were taken according to the number of carboxylic acid functional groups bound to the metal center (La, 4; Lu and Sc, 3; In, 2).

Interestingly, In-PYTA crystallized in the R3c space group as the 8-coordinate C₂-symmetric complex [In(H₂PYTA)]ClO₄ (FIG. 1). This result was somewhat unexpected because the ionic radius of In³⁺ is intermediate between that of Lu³⁺ and Sc³⁺ (Table 1). If the conformation of PYTA were driven exclusively by the size of the metal ion, the In-PYTA complex would be expected to crystallize in a configuration similar to that of Lu-PYTA and Sc-PYTA. Instead, as shown in FIG. 2, for the In³⁺ structure, two acetate groups of PYTA remain protonated and uncoordinated, and the macrocyclic backbone is folded nearly in half (N_{py}−M−N_{py}=108.58(5)°). This complex represents an extreme example of a twist-fold conformation for a py₂[18]dieneN₆ macrocycle. As the NMR data suggests, this conformation permits the macrocycle to be drawn closer to the metal center, leading to shorter average interatomic metal-donor distances than expected based on the ionic radius of In³⁺ (Table 1). This divergent configuration in comparison to the other complexes likely arises from differences in the aqueous chemistry of In³⁺ versus La³⁺, Lu³⁺, and Sc³⁺. Although all 4 ions are classified as relatively “hard” according to Pearson's hard-soft acid-base theory, the ionicity (I_{A}) of In³⁺, taken as the ratio of the electrostatic and covalent contributions to bonding, is lower than that of La³⁺, Lu³⁺, and Sc³⁺ (6.30 versus 10.30, 10.07, and 10.49, respectively). Moreover, In³⁺ typically forms complexes with lower coordination numbers ranging from 4 to 8 (H. R. Maecke et al., Nucl. Med., 1989, 30, 1235-1239). These properties may be expected to direct the configuration of the In-PYTA complex to one that cannot be predicted on the basis of ion size. As a final demonstration of the extreme versatility of PYTA, its crystal structure was obtained with Cu²⁺, a chemically softer, divalent metal ion with a 6-coordinate ionic radius of only 0.73 Å and an I_{A} of 2.68 (R. G. Pearson, J. Am. Chem. Soc., 1963, 85, 3533-3539). In this structure, all pendant arms remain unbound, and the nitrogen atoms of the macrocycle are arranged in a pseudo-octahedral geometry around the Cu center. This “twist-wrap” conformation of PYTA, shown in FIG. 2, is likewise adopted by the unfunctionalized py₂[18]dieneN₆ macrocycle when bound to Cu²⁺ (L. Branco et al., J. Chem. Soc., Dalton Trans., 2002, 3539-3550).

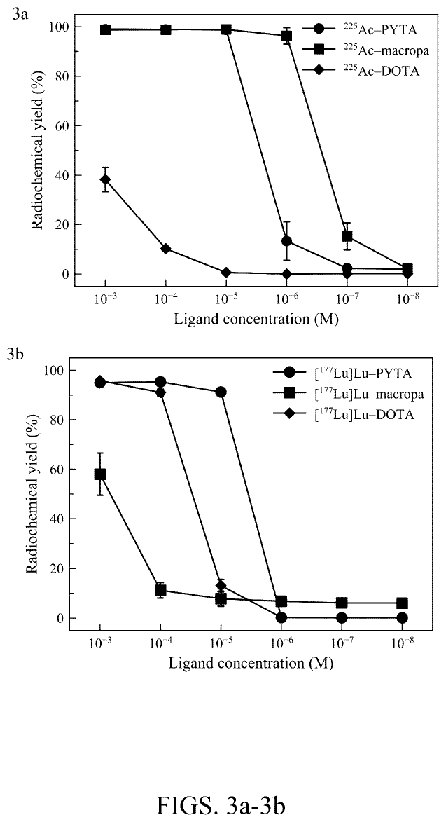

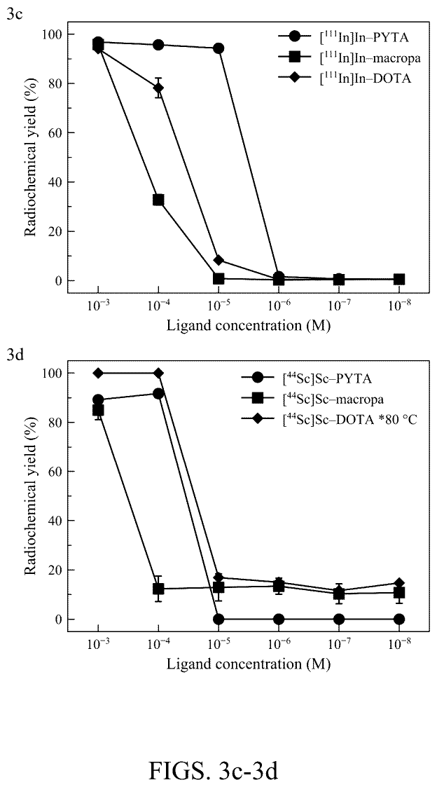

In view of the above results demonstrating that PYTA can accommodate a range of disparate metal ions by dramatically changing its conformation, next experiments sought to evaluate PYTA's ability to bind and stabilize ²²⁵Ac (t_{1/2}=10.0 d), ¹⁷⁷Lu (t_{1/2}=6.65 d), ¹¹¹In (t_{1/2}=2.80 d), and ⁴⁴Sc (t_{1/2}=3.97 h). Concentration-dependent radiolabeling experiments were performed in pH 6 NH₄OAc buffer at room temperature (RT). Radiochemical yields (RCYs) were assessed after 5 min and 60 min using radio-thin layer chromatography. To benchmark the performance of PYTA, additional radiolabeling experiments with DOTA and macropa were also conducted. Macropa, shown above, was included for comparison because of its rare ability to rapidly form stable complexes with ²²⁵Ac and other large radiometals at room temperature (N. A. Thiele et al., Angew. Chem., Int. Ed., 2017, 56, 14712-14717). The results of the radiolabeling studies are shown in the graphs in FIGS. 3a-3d (for ²²⁵Ac, ¹⁷⁷Lu, ¹¹¹In, and ⁴⁴S, respectively).

Significantly and unexpectedly, quantitative radiolabeling of PYTA was achieved with ²²⁵Ac, ¹⁷⁷Lu, and ¹¹¹In in just 5 minutes at chelator concentrations as low as 1×10⁻⁵ M. Likewise, PYTA was able to complex nearly all the ⁴⁴Sc at a chelator concentration of 1×10⁻⁴ M (and down to 4×10⁻⁵ M, measured at 60 min only). Notably, for all studies, a second minor peak was observed in the radio-TLCs of ⁴⁴Sc control samples regardless of the type of plate or mobile phase used. This small portion of activity, which remained unchelated even at the highest PYTA concentrations, has been attributed previously to the formation of insoluble (or difficult-to-chelate) oxide/hydroxide species (M. D. Phipps et al., Inorg. Chem., 2023, 62, 20567-20581). Increasing the mixing time to 60 min for ²²⁵Ac, ¹⁷⁷Lu, ¹¹¹In, and ⁴⁴Sc reactions did not give rise to higher RCYs, indicating that complexation equilibrium was reached rapidly for each radionuclide. Under the same conditions, DOTA was found to be a less effective chelator than PYTA for ²²⁵Ac, ¹⁷⁷Lu, and ¹¹¹In, even after allowing the reactions to proceed for 60 minutes and heating them to 80° C. This inferior labeling efficacy is reflected by the low RCYs of 1%, 44%, and 24% obtained for ²²⁵Ac, ¹⁷⁷Lu, and ¹¹¹In at 10⁻⁵ M chelator concentration, 60 min, and RT, conditions under which PYTA is quantitatively radiolabeled. Radiolabeling of DOTA with ⁴⁴Sc at 80° C. gave rise to RCYs on par with those obtained for PYTA but was observed to be inconsistent at room temperature. Similarly, the RCYs of macropa were also substantially lower than those of PYTA for ¹⁷⁷Lu, ¹¹¹In, and ⁴⁴Sc, but higher for ²²⁵Ac (FIGS. 3a-3d). These results are in line with macropa's exceptional ability to bind large metal ions like Ac³⁺ but correspondingly low affinity for smaller metal ions like Lu³⁺, In³⁺ and Sc³⁺.

Following these radiolabeling studies, the kinetic inertness of the resulting complexes of PYTA, DOTA, and macropa with ²²⁵Ac, ¹⁷⁷Lu, ¹¹¹In, and ⁴⁴Sc (PYTA only) were investigated in 75% human serum at 37° C. Human serum contains a variety of endogenous ligands and proteins that may compete with a chelator for metal binding, and therefore serves as a challenge to indicate the likelihood of transchelation in vivo. The percentages of intact complexes over time are compiled in Table 2 below.

TABLE 2
Stability (% intact complex remaining) measured at various time points after
incubating the ²²⁵Ac³⁺, [¹⁷⁷Lu]Lu³⁺, [¹¹¹In]In³⁺, and [⁴⁴Sc]Sc³⁺ complexes of PYTA,
macropa, and DOTA in 75% human serum at 37° C.^{a,b}

| | Before | | | | | | | | | | |
|---|---|---|---|---|---|---|---|---|---|---|---|
| | Serum | | | | | | | | | | |
| Complex | Addition | 1 h | 2 h | 8 h | 1 d | 2 d | 3 d | 4 d | 7 d | 14 d | 21 d |
| ²²⁵Ac-PYTA^{d} | 100 | 99 | ND | ND | 98 | 99 | 99 | 98 | 97 | 95 | 95 |
| ²²⁵Ac-macropa^{c,d} | 100 | 99 | ND | ND | 99 | 99 | 99 | ND | 99 | 98 | 97 |
| ²²⁵Ac-DOTA^{d} | 99 | 98 | ND | ND | 98 | 97 | 96 | ND | 94 | 89 | 88 |
| [¹⁷⁷Lu]Lu-PYTA^{d} | 100 | 99 | ND | ND | 99 | 99 | 98 | 98 | 99 | 98 | ND |
| [¹⁷⁷Lu]Lu-macropa^{e} | 82 | 0.6 | ND | ND | 0.9 | ND | ND | ND | ND | ND | ND |
| [¹⁷⁷Lu]Lu-DOTA^{f} | 99 | 98 | ND | ND | 99 | 99 | 98 | ND | 98 | 97 | ND |
| [¹¹¹In]In-PYTA^{d} | 97 | 97 | ND | ND | 97 | 95 | 96 | 97 | ND | ND | ND |
| [¹¹¹In]In-macropa^{g} | 99 | 95 | ND | ND | 87 | 91 | 93 | 92 | ND | ND | ND |
| [¹¹¹[In]In-macropa^{f} | 99 | 95 | ND | ND | 25 | ND | ND | ND | ND | ND | ND |
| [¹¹¹In]In-DOTA^{f} | 98 | 98 | ND | ND | 96 | 96 | 95 | 96 | ND | ND | ND |
| [⁴⁴Sc]Sc-PYTA^{d} | 94 | 94^{h} | 95 | 93 | ND | ND | ND | ND | ND | ND | ND |

^{a}Standard deviations for all values are #2%.
Bolded values indicate stability at the final time point.
^{b}ND = not determined.
^{c}Data taken from A. Hu et al., Inorg. Chem., 2022, 61, 12847-12855.
^{d}C_{chelator} = 1 × 10⁻⁵ M.
^{e}C_{chelator} = 1 × 10⁻³ M.
^{f}C_{chelator} = 1 × 10⁻⁴ M.
^{g}Cchelator = 8.4 × 10⁻⁴ M.
^{h}Measured at t = 30 min.

Significantly and unexpectedly, ²²⁵Ac-PYTA, [¹⁷⁷Lu]Lu-PYTA, [¹¹¹In]In-PYTA, and [⁴⁴Sc]Sc-PYTA all remained ≥95% intact in serum over the course of two half-lives of the respective radionuclides. Similar results were also observed for DOTA, which retained 88%, 97%, and 96% of ²²⁵Ac³⁺, [¹⁷⁷Lu]Lu³⁺ and [¹¹¹In]In³⁺ over the course of the study. It should be noted, however, that due to DOTA's subpar radiolabeling efficacy, the concentration of DOTA required in the ¹⁷⁷Lu and ¹¹¹In studies (10⁻⁴ M) to ensure complex formation at the start of the experiment was an order of magnitude higher than the concentration used for PYTA (10⁻⁵ M). Like PYTA and DOTA, macropa also formed a highly stable complex with ²²⁵Ac, releasing <3% of bound ²²⁵Ac over 21 d. By contrast, [¹⁷⁷Lu]Lu-macropa completely dissociated within the first hour of the experiment, and [¹¹¹In]In-macropa underwent 75% decomplexation within 1 d. Interestingly, when a higher concentration of macropa (8.4×10⁻⁴ M vs. 1×10⁻⁴ M) was used in the ¹¹¹In serum challenge, a dramatic rise in complex stability to 92% at 4 d was noted. This increase in intact complex with increasing chelator concentration is likely a thermodynamic effect arising from metal-ligand recombination in solution in the closed-tube system used for these studies, rather than a reflection of enhanced kinetic stability. Collectively, the radiolabeling studies and serum challenges reveal PYTA to be far superior to DOTA and macropa for rapidly binding and stabilizing this “superfecta” of medical radionuclides under mild, physiologically relevant conditions.

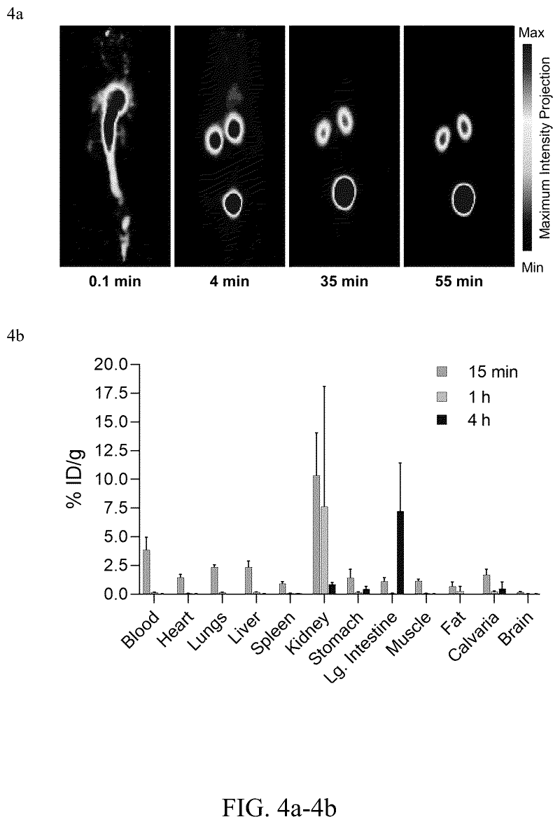

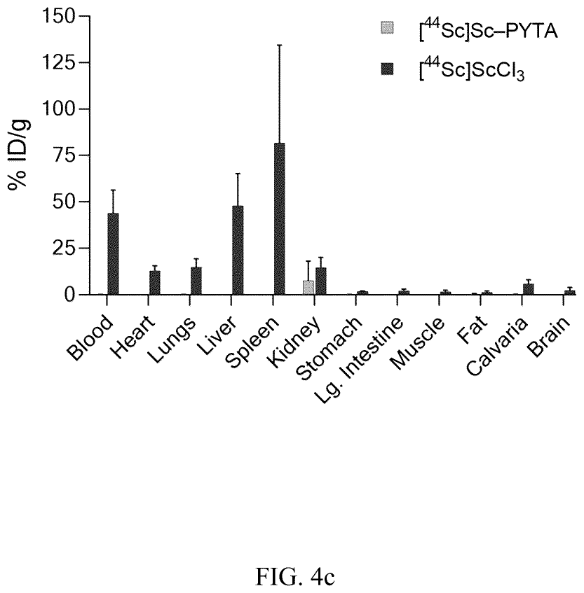

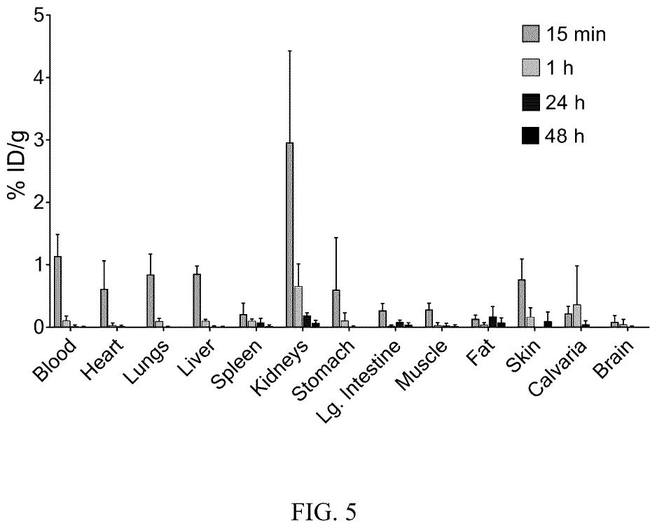

Finally, to establish the potential use of PYTA for in vivo diagnostic and therapeutic applications, imaging and biodistribution studies in mice with [⁴⁴Sc]Sc-PYTA and ²²⁵Ac-PYTA were performed. These complexes have the most extreme disparity with respect to metal-ion size. For ⁴⁴Sc studies, healthy female Swiss Webster mice were injected with [⁴⁴Sc]Sc-PYTA (~5.18 MBq) and monitored by dynamic PET imaging over the course of 55 min. Groups of mice were also sacrificed at 15 min, 1 h, and 4 h post injection, and the activity in their organs were quantified ex vivo. Frames across the dynamic PET acquisition, as shown in FIG. 4a, revealed uptake and localization of activity primarily in the kidneys and bladder of mice, indicative of rapid renal clearance of the intact complex. Additionally, a small amount of intestinal uptake was noted at these early time points, suggesting that some excretion also occurs through the hepatobiliary pathway. The biodistribution profile of [⁴⁴Sc]Sc-PYTA, as shown in FIG. 4b, corroborated the results of the PET imaging, showing high kidney uptake at 15 min (10.3% ID per g) that diminished over time as the complex was excreted, and a spike in intestinal activity (7.2% ID per g) at 4 h post injection. Interestingly, this gut uptake is substantially less than that observed previously for other radio-scandium complexes, such as [^{43/47}Sc]Sc—HOPO (~19% ID per g at 4 h), which highlights the different pharmacokinetic properties of these complexes.

Of further significance, as shown in the data in FIG. 4c, the biodistribution profile of [⁴⁴Sc]Sc-PYTA differs dramatically from that of free ⁴⁴Sc (administered as [⁴⁴Sc]ScCl₃), which exhibits a long circulation time and substantial uptake in the heart, lungs, liver, and spleen of mice (FIG. 4c)(N. Benabdallah et al., EJNMAMI Res., 2023, 13, 17). Thus, the absence of accumulation of activity in these organs upon administering [⁴⁴Sc]Sc-PYTA to mice signifies that PYTA is able to retain ⁴⁴Sc in vivo over the time course of the study. As shown by the data in FIG. 5, similar biodistribution results were obtained upon administering ²²⁵Ac-PYTA (~3.7 kBq) to mice. Specifically, the complex was rapidly cleared through the renal system, and no activity was observed to accumulate in any organ over 48 h. This distribution pattern contrasts with that of free ²²⁵Ac³⁺, which is taken up by the liver, spleen, and bone of mice (N. A. Thiele et al., Angew. Chem., Int. Ed., 2017, 56, 14712-14717). The predominant renal clearance of [⁴⁴Sc]Sc-PYTA and ²²⁵Ac-PYTA observed here in mice is similar to the biodistribution of [¹⁵3Sm]Sm-PYTA observed previously in rats (L. Miao et al., Inorg. Chem., 1995, 34, 2233-2243). Collectively, the results of these in vivo studies establish proof of concept for PYTA as a robust chelator for a complementary set of medically important radionuclides.

### Synthesis and Characterization of Bifunctional PYTA

An isothiocyanate (NCS) functionalized version of PYTA was synthesized in accordance with the following synthetic scheme:

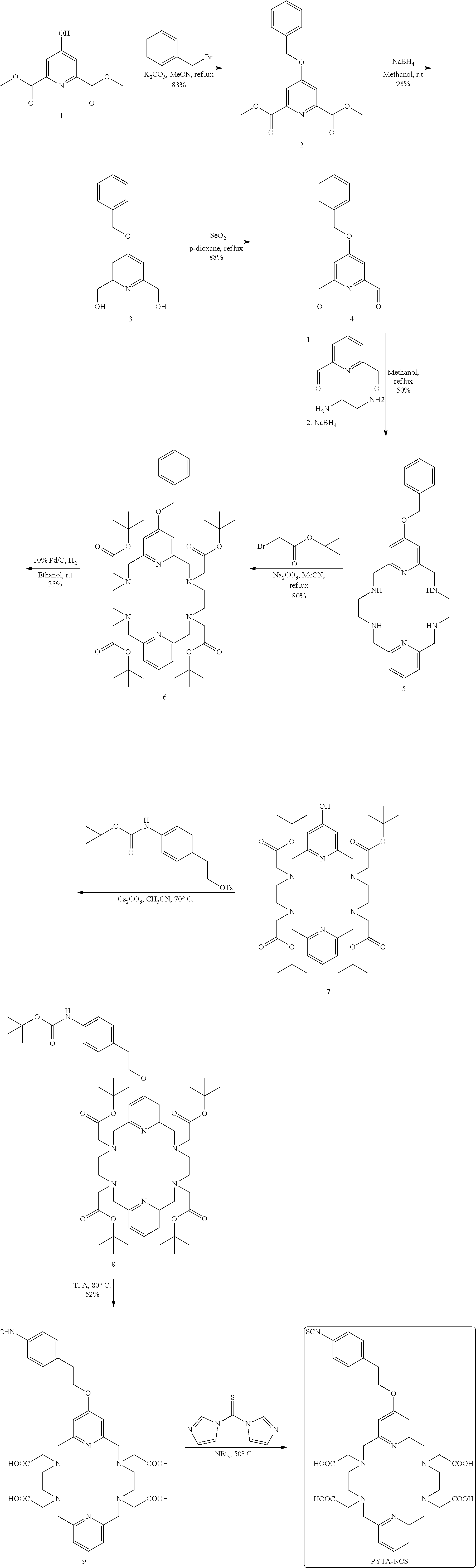

### Synthesis of dimethyl 4-(benzyloxy)pyridine-2,6-dicarboxylate (Compound 2)

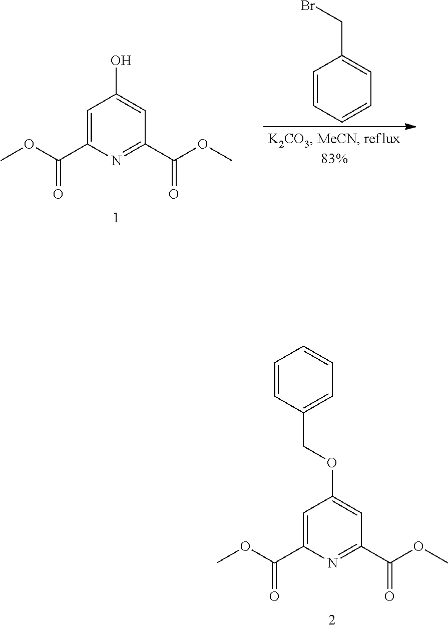

Compound 2 was synthesized according to a previously reported method (Li et al., Dalton Trans. 2020, 49, 5547-5562). Briefly, in a round-bottom flask, compound 1 (5.00 g, 10.910 mmol) was dissolved in CH₃CN (100 mL). Then, potassium carbonate (15.780 g, 54.551 mmol) was added. The mixture was then heated to reflux for 2 h. After 2 h, the reaction mixture was cooled to room temperature. Then, benzyl bromide (9.331 g, 27.261 mmol) was dissolved in 30 mL of CH₃CN and added to the suspension. The reaction mixture was then heated to reflux and stirred for 18 h. Upon completion, the reaction was cooled to room temperature and the pale-yellow suspension was then filtered to remove excess solids. The filtrate was concentrated at 50° C. on the rotary evaporator to a brown oil. The yellow oil was dissolved in 100 mL of dichloromethane and washed with H₂O (50 mL×3). The organic layer was dried over sodium sulfate and concentrated at 50° C. on the rotary evaporator to give a crude brown oil. The crude material was then purified on normal phase silica using flash column chromatography (100% Hexanes→50% Hexanes: 50% Ethyl Acetate), yielding the title Compound 2 as a white solid (5.014 g, 84%). ¹H NMR (400 MHz, DMSO-d₆) δ 7.84 (s, 2H), 7.49 (d, J=7.4 Hz, 2H), 7.39 (m, 3H), 5.38 (s, 2H), 3.90 (s, 6H). ¹³C NMR (101 MHz, DMSO-d₆) δ 166.17, 164.58, 149.36, 135.48, 128.57, 128.29, 127.86, 114.45, 70.20, 52.69.

### Synthesis of (4-(benzyloxy)pyridine-2,6-diyl)dimethanol (Compound 3)

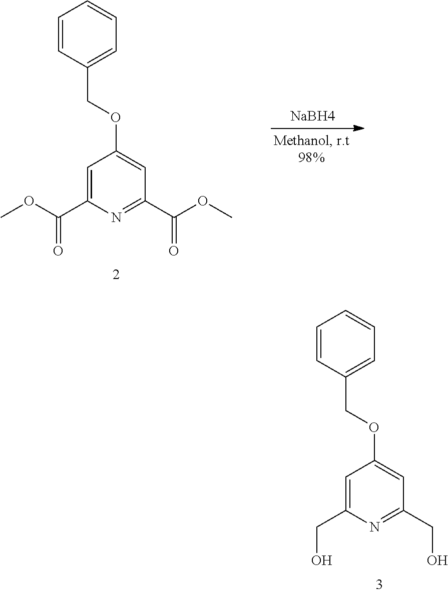

Compound 3 was synthesized according to a previously reported method (Li et al., Ibid.). In a round-bottom flask, Compound 2 (4.711 g, 15.64 mmol) was dissolved in dry MeOH (60 mL). The solution was then placed in an ice bath and sodium borohydride (NaBH₄) (1.774 g, 46.92 mmol) was added in three portions over 1 h. The reaction mixture was stirred at room temperature for 18 h. The mixture was then diluted with CHCl₃ (50 mL) and then quenched with saturated aqueous NaHCO₃ solution (40 mL). The organic phase was removed at 50° C. on rotary evaporator. The aqueous solution was then extracted with CHCl₃ (100 mL×3). The combined organic phases were dried over anhydrous magnesium sulfate (MgSO₄), and then filtered. The filtrate was concentrated on the rotary evaporator to give the product (3) as white solid (3.774 g, 98%). ¹H NMR (400 MHz, DMSO-d₆) δ 7.46 (d, J=7.4 Hz, 2H), 7.41 (t, J=7.6 Hz, 2H), 7.35 (t, J=7.1 Hz, 1H), 6.94 (s, 2H), 5.41-5.32 (m, 2H), 5.20 (s, 2H), 4.46 (d, J=4.7 Hz, 4H), 2.50 (p, J=1.9 Hz, 2H). ¹³C NMR (101 MHz, DMSO-d₆) δ 165.84, 163.08, 136.41, 128.56, 128.08, 127.72, 104.79, 69.10, 64.06.

### Synthesis of 4-(benzyloxy)pyridine-2,6-dicarbaldehyde (Compound 4)

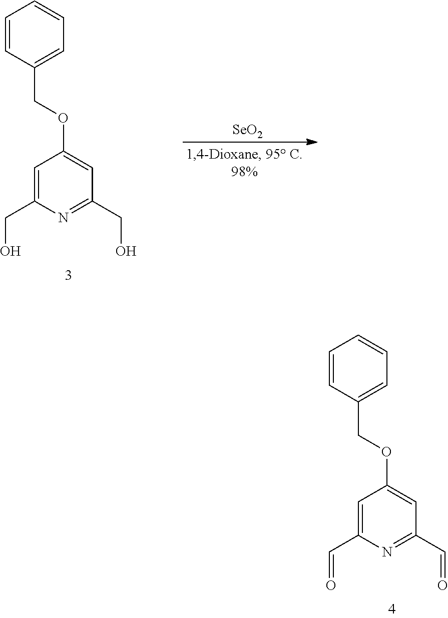 [SMILES: OCc1cc(OCc2ccccc2)cc(CO)n1 ; "SeO2" ; O=Cc1cc(OCc2ccccc2)cc(C=O)n1]

Compound 4 was synthesized according to a previously reported method (A. Hu et al., Eur. J. Inorg. Chem. 2023, 26, e202300457). In a 250 mL, Compound 3 (7.902 g, 32.21 mmol) was dissolved in 100 mL of dry p-dioxane. Then, SeO₂ (8.934 g, 80.52 mmol) was added, and the suspension was heated at 95° C. for 3 h. The completion of the reaction was monitored using thin layer chromatography (TLC). Once completed, the reaction mixture was cooled to r.t. and then filtered using 45 μm nylon filter. The filtrate was concentrated to dryness under reduced pressure, giving a pink solid. The solids were then dissolved in CH₂Cl₂ and kept into the refrigerator (7° C.). The solids were then filtered off and the filtrate was dried under vacuum to yield the product (4) as orange solids (6.837 g, 88%). ¹H NMR (400 MHz, DMSO-d₆) δ 10.04 (s, 2H), 7.76 (s, 2H), 7.49 (d, J=7.2 Hz, 2H), 7.39 (m, 3H), 5.41 (s, 2H). ¹³C NMR (101 MHz, DMSO-d₆) δ 192.56, 166.52, 154.46, 135.41, 128.62, 128.36, 127.95, 111.99, 70.42.

### Synthesis of 1⁴-(benzyloxy)-3,6,10,13-tetraaza-1,8(2,6)-dipyridinacyclotetradecaphane (Compound 5)

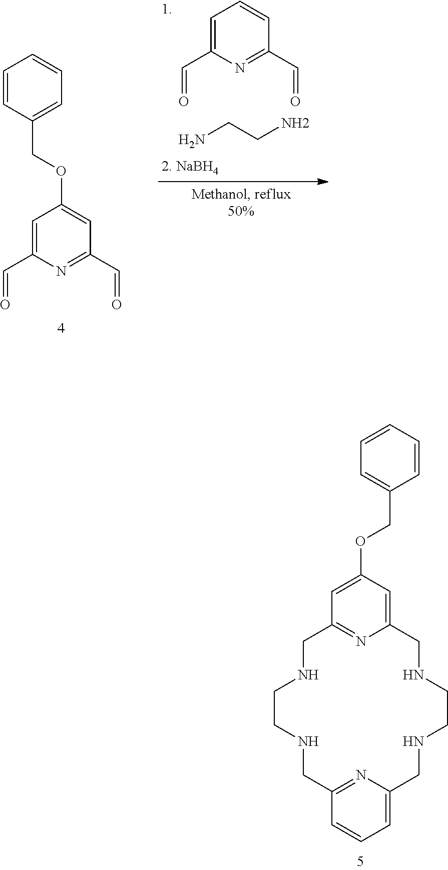

In a 500 mL round bottom flask, ethylenediamine (0.278 mL, 4.145 mmol) was mixed with 10 mL of dry methanol. The solution was then heated to reflux. Then, 2,6-pyridinedicarbaldehyde (0.280 g, 2.073 mmol, dissolved in 120 mL dry methanol) was added dropwise to the solution over 6 h using a syringe pump (20 mL/hr). After the addition was complete, BaCl₂·2H₂O (0.506 g, 2.073 mmol) was added to the reaction mixture. Then, the reaction mixture was heated to reflux. To the stirred reaction mixture, Compound 4 (0.500 g, 2.073 mmol, dissolved in 120 mL dry methanol) was added dropwise over 12 h (10 mL/h) using a syringe pump. Upon addition, the rection mixture was allowed to cool to room temperature. The flask was then placed in an ice bath, solid NaBH₄ (0.470 g, 12.435 mmol) was added slowly and stirred for 2 h. Completion of the reaction was confirmed using analytical HPLC and LR-MS. Once completed, NaBH₄ was quenched using sat. NaHCO₃ solution (50 mL). Then, methanol was removed under reduced pressure and the remaining aqueous phase was extracted using CHCl₃ (100 mL×3). Then, the combined organics were dried over MgSO₄ and filtered. The filtrate was concentrated under reduced pressure to yield crude yellow oil. The yellow oil was dissolved in H₂O containing 10% CH₃OH and 0.1% TFA (20 mL) and injected through the preparative HPLC system to purify the product (Method: 0-10 min, 90% H₂O/CH₃OH; 10-40 min, 90%→0% H₂O/CH₃OH). Pure fractions were concentrated under reduced pressure and then lyophilized to yield the final product (5) as slight yellow flaky solids (0.947 g, 50%). ¹H NMR (400 MHz, DMSO-d₆) δ 9.40 (s, 4H), 8.01 (t, J=7.8 Hz, 1H), 7.54 (d, J=7.8 Hz, 2H), 7.48-7.35 (m, 5H), 7.24 (s, 2H), 5.25 (s, 2H), 4.49 (d, J=35.8 Hz, 8H), 3.44 (s, 8H). ¹³C NMR (101 MHz, DMSO-d₆) δ 166.28, 159.96, 159.63, 159.29, 152.83, 151.16, 139.12, 135.68, 128.79, 128.58, 128.22, 122.58, 121.12, 118.19, 115.25, 112.31, 109.31, 70.14, 50.39, 43.39. ESI HR-MS: Found: m/z: 433.2711. Calculated for [C₂₅H₃₂N₆O+H]⁺: m/z 433.2711. Elemental Analysis: Found %: C 43.48, H 4.30, F 24.85, N 9.11. Calculated % for C₂₅H₃₂N₆O·4TFA 1.5H₂O: C 43.29, H 4.29, F 24.90, N 9.18, O 18.35. Analytical HPLC: t_{R}=15.94 min. Method: 0-5 min, 10% MeOH/H₂O with 0.1% TFA; 5-25 min, linear gradient from 10-100% MeOH/H₂O with 0.1% TFA, 25-30 min, 100% MeOH.

### Synthesis of tetra-tert-butyl 2,2′,2″,2′″-(1⁴-(benzyloxy)-3,6,10,13-tetraaza-1,8(2,6)-dipyridinacyclotetradecaphane-3,6,10,13-tetrayl)tetraacetate (Compound 6)

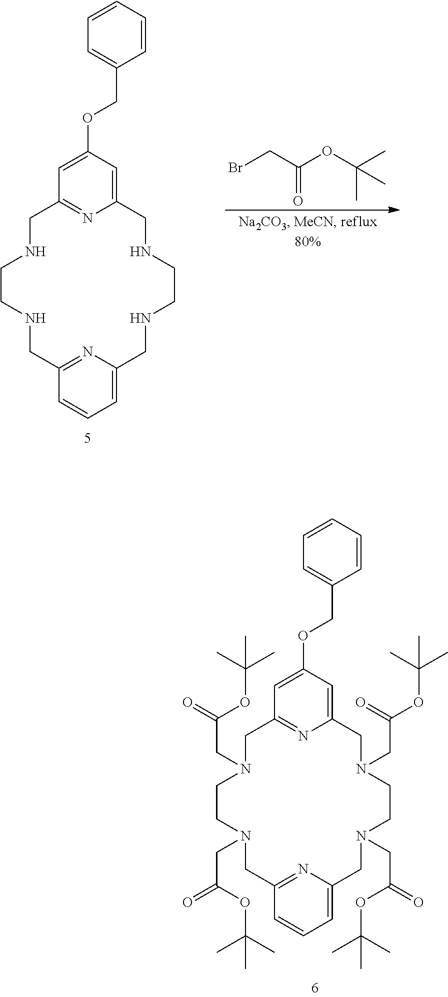

In a 500 mL RB flask, 5 (3.201 g, 7.397 mmol) was dissolved in 150 mL of MeCN. The Na₂CO₃ (9.408 g, 105.988 mmol) was added and heated to reflux for 2 h. After 2 h, tert-butyl bromoacetate (6.205 g, 31.809 mmol) was added. The suspension was stirred at reflux for 16 h and then filtered. The filtrate was concentrated in vacuo to obtain a light yellow oil. The crude oil was further purified using flash column chromatography with normal phase silica (100% CH₂Cl₂→90% CH₂Cl₂:10% CH₃OH) to yield the product 6 as waxy yellow solid (5.262 g, 80%). ¹H NMR (400 MHz, CDCl₃) δ 7.65 (t, J=7.7 Hz, 1H), 7.40-7.28 (m, 5H), 7.12 (d, J=7.7 Hz, 2H), 6.76 (s, 2H), 5.10 (s, 2H), 3.85 (d, J=22.7 Hz, 8H), 3.02 (s, 8H), 2.84 (s, 8H), 1.28 (d, J=4.1 Hz, 36H). ¹³C NMR (101 MHz, CDCl₃) δ 171.41, 171.26, 166.11, 159.43, 157.64, 137.96, 135.38, 128.61, 128.30, 127.60, 122.42, 109.16, 81.63, 81.61, 77.48, 76.84, 70.17, 57.54, 57.22, 56.19, 52.65, 50.31, 27.98. ESI-HRMS: Found: m/z 889.5430. Calculated for [C₄₉H₇₂N₆O₉+H]⁺: m/z 889.5434. Analytical HPLC: t_{R}=22.33 min Method: 0-5 min, 10% MeCN/H₂O with 0.1% TFA; 5-25 min, linear gradient from 10-100% MeCN/H₂O with 0.1% TFA, 25-30 min, 100% MeCN.

### Synthesis of tetra-tert-butyl 2,2′,2″,2′″-(1⁴-hydroxy-3,6,10,13-tetraaza-1,8(2,6)-dipyridinacyclotetradecaphane-3,6,10,13-tetrayl)tetraacetate (Compound 7)

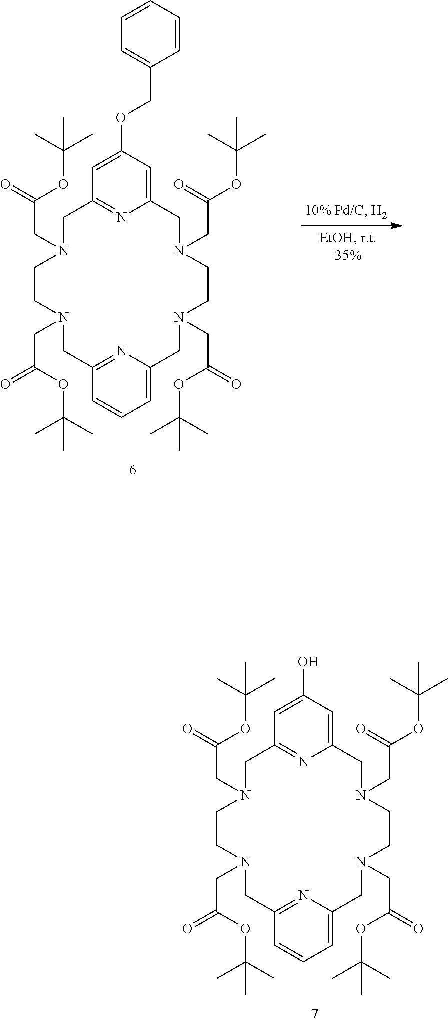 [SMILES: CC(C)(C)OC(=O)CN1CCN(CC(=O)OC(C)(C)C)Cc2cc(OCc3ccccc3)cc(n2)CN(CC(=O)OC(C)(C)C)CCN(CC(=O)OC(C)(C)C)Cc2cccc(n2)C1 ; CC(C)(C)OC(=O)CN1CCN(CC(=O)OC(C)(C)C)Cc2cc(O)cc(n2)CN(CC(=O)OC(C)(C)C)CCN(CC(=O)OC(C)(C)C)Cc2cccc(n2)C1]

In a round bottom flask, Compound 6 (1.412 g, 1.588 mmol) was dissolved in 100 mL of ethanol and 10% Pd/C, type 487 (0.248 g, 2.337 mmol) was added, and the mixture was stirred under H₂ atmosphere at r.t. The reaction progress was monitored by analytical HPLC and LR-MS. After the reaction was complete, the suspension was filtered, and the filtrate was concentrated to dryness at 50° C., yielding a yellow oil. This crude material was then dissolved in H₂O containing 10% CH₃OH and 0.1% TFA (10 mL). Following filtration, this solution was injected into the preparative HPLC system to purify the product (Method: 0-10 min, 90% H₂O/CH₃OH; 10-35 min, 90%→0% H₂O/CH₃OH). Pure fractions were combined, concentrated under reduced pressure, and lyophilized to yield 7 as a white solid (0.444 g, 35%). ESI-HRMS m/z: 799.4962; calcd m/z for [C₄₂H₆₆N₆O₉+H]⁺: 799.4965. Analytical HPLC: t_{R}=23.05 min Method: 0-5 min, 10% MeOH/H₂O with 0.1% TFA; 5-25 min, linear gradient from 10-100% MeOH/H₂O with 0.1% TFA, 25-30 min, 100% MeOH.

### Synthesis of tetra-tert-butyl 2,2′,2″,2′″-(1⁴-(4-((tert-butoxycarbonyl)amino)phenethoxy)-3,6,10,13-tetraaza-1,8(2,6)-dipyridinacyclotetradecaphane-3,6,10,13-tetrayl)tetraacetate (Compound 8)

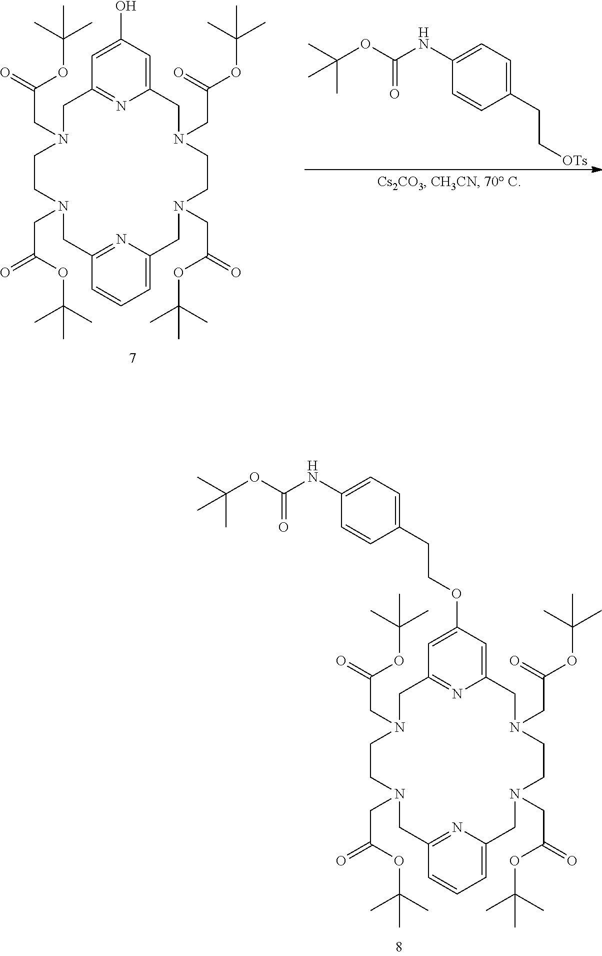 [SMILES: CC(C)(C)OC(=O)CN1CCN(CC(=O)OC(C)(C)C)Cc2cc(O)cc(n2)CN(CC(=O)OC(C)(C)C)CCN(CC(=O)OC(C)(C)C)Cc2cccc(n2)C1 ; Cc1ccc(S(=O)(=O)OCCc2ccc(NC(=O)OC(C)(C)C)cc2)cc1 ; CC(C)(C)OC(=O)CN1CCN(CC(=O)OC(C)(C)C)Cc2cc(OCCc3ccc(NC(=O)OC(C)(C)C)cc3)cc(n2)CN(CC(=O)OC(C)(C)C)CCN(CC(=O)OC(C)(C)C)Cc2cccc(n2)C1]

To a mixture of 7 (0.230 g, 0.287 mmol) and Cs₂CO₃ (0.563 g, 1.728 mmol), dry CH₃CN (10 mL) was added. This mixture was heated at 70° C. for 1 h. Then, 4-((tert-butoxycarbonyl)amino)phenethyl 4-methylbenzenesulfonate (0.175 g, 0.448 mmol) was added. This reaction mixture was then heated at 70° C. for 48 h. Afterwards, the suspension was filtered, and the filtrate was concentrated to dryness under reduced pressure, giving a white solid. This crude material containing the desired compound 8, was directly used in the next step. LRMS measured m/z: 1018.6; calcd for [C₅₅H₈₃N₇O₁₁]⁺: 1018.62.

### Synthesis of 2,2′,2″,2′″-(1⁴-(4-aminophenethoxy)-3,6,10,13-tetraaza-1,8(2,6)-dipyridinacyclotetradecaphane-3,6,10,13-tetrayl)tetraacetic acid (Compound 9)

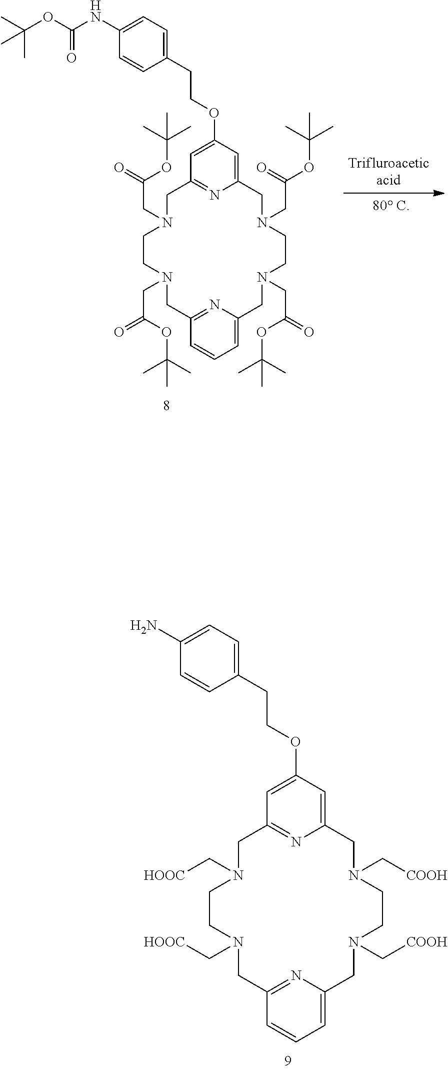 [SMILES: CC(C)(C)OC(=O)CN1CCN(CC(=O)OC(C)(C)C)Cc2cc(OCCc3ccc(NC(=O)OC(C)(C)C)cc3)cc(n2)CN(CC(=O)OC(C)(C)C)CCN(CC(=O)OC(C)(C)C)Cc2cccc(n2)C1 ; Nc1ccc(CCOc2cc3nc(c2)CN(CC(=O)O)CCN(CC(=O)O)Cc2cccc(n2)CN(CC(=O)O)CCN(CC(=O)O)C3)cc1]

The crude material from the last step was dissolved in TFA (10 mL), and this mixture was heated at 80° C. for 16 h, during which the tert-butyloxycarbonyl (Boc) protecting group was removed. The completion of the reaction was monitored using analytical HPLC and LRMS. Once completed, the TFA was then removed under reduced pressure, and the resulting residue was dissolved in H₂O containing 10% CH₃OH and 0.1% TFA (4 mL). This solution was injected into the preparative HPLC system to purify the product (Method: 0-5 min, 90% H₂O/CH₃OH; 5-35 min, 90%-0% H₂O/CH₃OH). Pure fractions were combined, concentrated under reduced pressure, and lyophilized to yield product, 9 as a white solid (0.101 g, 52% from 7). ¹H NMR (400 MHz, DMSO-d₆) δ 7.84 (t, J=7.7 Hz, 1H), 7.45 (dd, J=17.2, 7.9 Hz, 3H), 7.25 (d, J=8.1 Hz, 2H), 7.20 (s, 2H), 7.11 (d, J=8.1 Hz, 1H), 6.97 (s, 2H), 4.32 (d, J=18.4 Hz, 6H), 4.13 (s, 4H), 3.75 (s, 4H), 3.55 (s, 4H), 3.24 (s, 4H), 3.13 (s, 4H), 3.03 (t, J=6.9 Hz, 2H). LRMS measured m/z: 693.9; calcd m/z for [C₃₄H₄₃N₇O₉]⁺: 693.76. Analytical HPLC: t_{R}=12.91 min Method: 0-5 min, 10% MeOH/H₂O with 0.1% TFA; 5-25 min, linear gradient from 10-100% MeOH/H₂O with 0.1% TFA, 25-30 min, 100% MeOH.

### Synthesis of 2,2′,2″,2′″-(1⁴-(4-isothiocyanatophenethoxy)-3,6,10,13-tetraaza-1,8(2,6)-dipyridinacyclotetradecaphane-3,6,10,13-tetrayl)tetraacetic acid (PYTA-NCS)

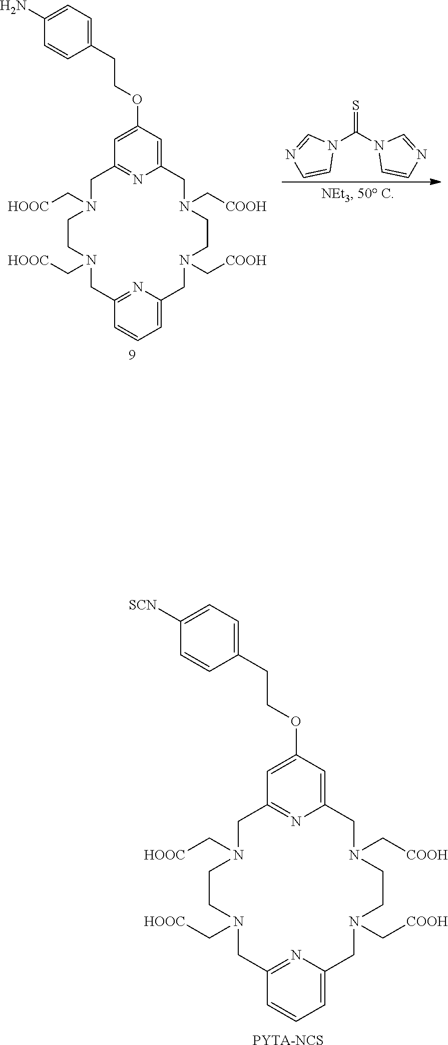

In a 50 mL round bottom flask, Compound 9 (50 mg, 0.072 μmol) was dissolved in 3 mL of DMF. Then, triethylamine (50 μL, 0.3605 μmol) was added to the solution and the mixture was stirred for 30 min. After 30 min, 1,1′-thiocarbonyldiimidazole (17 mg, 0.0937 μmol) was added to the reaction and the mixture was stirred at 50° C. for 16 h. The reaction was monitored using LRMS. Once complete, the reaction mixture was cooled to RT. Once cooled, the solution was diluted with diethyl ether (30 mL) and kept at −20° C. for 2 h to induce precipitation. The crude product was isolated via centrifugation and washed with diethyl ether (2×20 mL). The resultant ppt was then purified using preparative HPLC to yield the final product. LRMS measured m/z: 736.0, Calcd m/z [C₃₅H₄₁N₇O₉S]⁺. 735.81.

While there have been shown and described what are at present considered the preferred embodiments of the invention, those skilled in the art may make various changes and modifications which remain within the scope of the invention defined by the appended claims.

What is claimed is:

1. A macrocycle-radionuclide complex comprising the following formula: 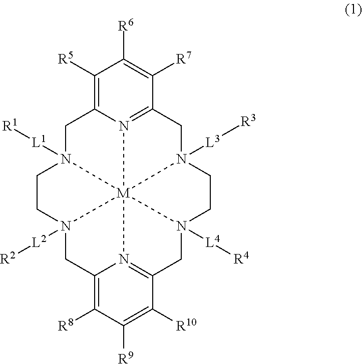
wherein:
L¹, L², L³, and L⁴ are independently selected from a linker of the formula (CH₂)ₚ, wherein p is an integer of 0, 1, 2, or 3;
R¹, R2, R3, and R4 are independently selected from: (i) oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt; (ii) aryl groups; and (iii) heteroaryl groups;
R⁵, R⁶, R⁷, R⁸, R⁹, and R¹⁰ are independently selected from: (i) H; (ii) hydrophilic groups of the formula L^{a}_{z}-R^{a}, wherein L^{a} is a hydrocarbon linker containing 1-12 carbon atoms, z is 0 or 1, and R^{a} is independently selected from the group consisting of —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂; and (iii) highly reactive groups of the formula L^{b}_{z}-R^{b}, wherein L^{b} is a hydrocarbon linker containing 1-12 carbon atoms and optionally containing one or more heteroatoms selected from O, N, and S; z is 0 or 1; and R^{b} is independently selected from the group consisting of —OCN, —SCN, —NCO, —NCS, —NR′—NR′₂, —NR′₂, —N═C═N—R′, —SH, —SSR′, —SO₂Cl, —C(O)Cl, alkyne-containing groups, alkene-containing groups, azide-containing groups, aldehyde-containing groups, tetrazine-containing groups, activated ester groups, tetrafluorophenyl, maleimide groups, and epoxide groups; and
M is a radionuclide atom useful in diagnostic imaging or cancer therapy.

2. The complex of claim 1, wherein M is selected from the group consisting of ¹⁷⁷Lu³⁺, ⁴³Sc³⁺, ⁴⁴Sc³⁺, ⁴⁷Sc³⁺, ⁶⁶Ga³⁺, ⁶⁷Ga³⁺, ⁶⁸Ga³⁺, ⁸⁶Y³⁺, ⁸⁸Y³⁺, ⁹⁰Y³⁺, ⁸⁹Zr⁴⁺, ¹¹¹In³⁺, ¹¹⁰In³⁺, ^{114m}In³⁺, ¹³⁵La³⁺, ¹³²La³⁺, ¹³⁴La³⁺, ¹³⁴Ce³⁺, ¹³⁷Ce³⁺, ¹⁵²Dy³⁺, ¹⁴⁹Tb³⁺, ¹⁵²Tb³⁺, ¹⁵⁵Tb³⁺, ¹⁶¹Tb³⁺, ²¹³Bi³⁺, ²¹²Bi³⁺, ²²⁷Th⁴⁺, ²²⁶Th⁴⁺, ²²⁵Ac³⁺, ²²⁶Ac³⁺, ²²³Ra²⁺, ²²⁴Ra²⁺, ¹³¹Ba²⁺, ²¹²Pb²⁺, ²¹²Pb⁴⁺, ²⁵⁵Fm³⁺, ²³⁰U⁶⁺, ²³⁰U⁴⁺, ²⁰¹Tl, ⁹⁰Nb, ¹⁴⁹Pm³⁺, ²³⁹Np⁴⁺, ²³⁹Np⁵⁺, ¹¹⁹Sb³⁺, ¹¹⁷Sb³⁺, ¹¹⁹Sb⁵⁺, ¹¹⁷Sb⁵⁺, ^{52g}Mn, ⁶⁴Cu, ⁴⁵Ti⁴⁺, ⁵⁵Co, and ^{58m}Co.

3. The complex of claim 1, wherein M is selected from the group consisting of ²²⁵Ac³⁺, ²²⁶Ac³⁺, ¹⁷⁷Lu³⁺, ¹¹¹In³⁺, ¹¹⁰In³⁺, ^{114m}In³⁺, ⁴³Sc³⁺, ⁴⁴Sc³⁺, and ⁴⁷Sc³⁺.

4. The complex of claim 1, wherein R⁵, R⁶, R⁷, R⁸, R⁹, and R¹⁰ are all H and the complex has the following formula:

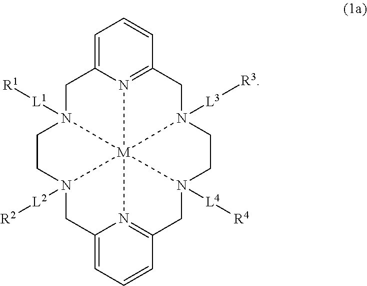

5. The complex of claim 1, wherein the complex has the following formula:

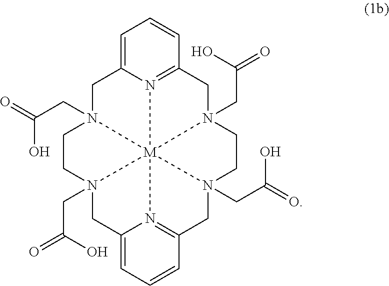

6. The complex of claim 5, wherein M is selected from the group consisting of ²²⁵Ac³⁺, ²²⁶Ac³⁺, ¹⁷⁷Lu³⁺, ¹¹¹In³⁺, ^{110m}In³⁺, ^{114m}In³⁺, ⁴³Sc³⁺, ⁴⁴Sc³⁺ and ⁴⁷Sc³⁺.

7. The complex of claim 1, wherein at least one of R⁵, R⁶, R⁷, R⁸, R⁹, and R¹⁰ is a group independently selected from groups (ii) and (iii).

8. The complex of claim 1, wherein at least one of R⁵, R⁶, R⁷, R⁸, R⁹, and R¹⁰ is a highly reactive group selected from groups recited in group (iii).

9. The complex of claim 1, wherein the complex has the following formula: 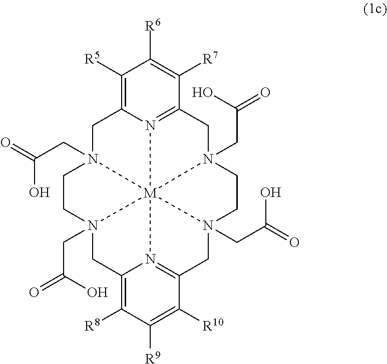
wherein at least one of R⁵, R⁶, R⁷, R⁸, R⁹, and R¹⁰ is a group independently selected from groups (ii) and (iii).

10. The complex of claim 9, wherein at least one of R⁵, R⁶, R⁷, R⁸, R⁹, and R¹⁰ is a highly reactive group selected from groups recited in group (iii).

11. A chelator molecule having the following formula: 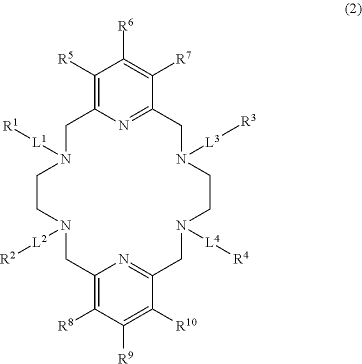
wherein:
L¹, L², L³, and L⁴ are independently selected from a linker of the formula (CH₂)ₚ, wherein p is an integer of 0, 1, 2, or 3;
R¹, R², R³, and R⁴ are independently selected from: (i) oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt; (ii) aryl groups; and (iii) heteroaryl groups;
R⁵, R⁶, R⁷, R⁸, R⁹, and R¹⁰ are independently selected from: (i) H; (ii) hydrophilic groups of the formula L^{a}_{z}-R^{a}, wherein L^{a} is a hydrocarbon linker containing 1-12 carbon atoms, z is 0 or 1, and R^{a} is independently selected from the group consisting of —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂; and (iii) highly reactive groups of the formula L^{b}_{z}-R^{b}, wherein L^{b} is a hydrocarbon linker containing 1-12 carbon atoms and optionally containing one or more heteroatoms selected from O, N, and S; z is 0 or 1; and R^{b} is independently selected from the group consisting of —OCN, —SCN, —NCO, —NCS, —NR′—NR′₂, —NR′₂, —N═C═N—R′, —SH, —SSR′, —SO₂Cl, —C(O)Cl, alkyne-containing groups, alkene-containing groups, azide-containing groups, aldehyde-containing groups, tetrazine-containing groups, activated ester groups, tetrafluorophenyl, maleimide groups, and epoxide groups wherein at least one of R⁵, R⁶, R⁷, R⁸, R⁹, and R¹⁰ is a non-H group selected from groups (ii) and (iii).

12. The chelator molecule of claim 11, wherein at least one of R⁵, R⁶, R⁷, R⁸, R⁹, and R¹⁰ is a highly reactive group selected from groups recited in group (iii).

13. The chelator molecule of claim 11, wherein the chelator molecule has the following formula: 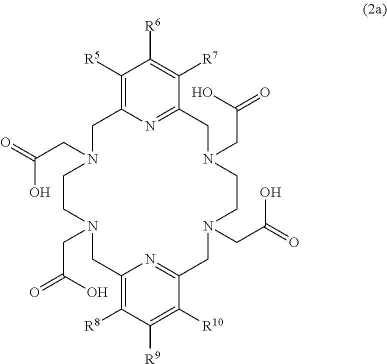
wherein at least one of R⁵, R⁶, R⁷, R⁸, R⁹, and R¹⁰ is a group independently selected from groups (ii) and (iii).

14. The chelator molecule of claim 13, wherein at least one of R⁵, R⁶, R⁷, R⁸, R⁹, and R¹⁰ is a highly reactive group selected from groups recited in group (iii).

15. A cancer cell-targeting radionuclide complex comprising the following formula: 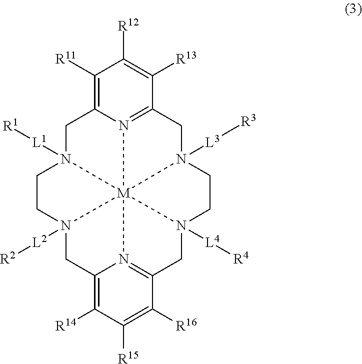
wherein:
L¹, L², L³, and L⁴ are independently selected from a linker of the formula (CH₂)ₚ, wherein p is an integer of 0, 1, 2, or 3;
R¹, R², R³, and R⁴ are independently selected from: (i) oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt; (ii) aryl groups; and (iii) heteroaryl groups;
R¹¹, R¹², R¹³, R¹⁴, R^{1S}, and R¹⁶ are independently selected from: (i) H; (ii) hydrophilic groups of the formula L^{a}_{z}-R^{a}, wherein L^{a} is a hydrocarbon linker containing 1-12 carbon atoms, z is 0 or 1, and R^{a} is independently selected from the group consisting of —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂; and (iii) cancer cell targeting groups of the formula L^{c}_{z}-R^{c}, wherein L^{c} is a hydrocarbon linker containing 1-12 carbon atoms and optionally containing one or more heteroatoms selected from O, N, and S, or L^{c} is a linking particle; z is 0 or 1; and R^{c} is a cancer cell targeting agent; wherein at least one of R¹¹, R¹², R¹³, R¹⁴, R¹⁵, and R¹⁶ is a cancer cell targeting group L^{c}_{z}-R^{c}; and
M is a radionuclide atom useful in diagnostic imaging or cancer therapy.

16. The complex of claim 15, wherein the biological targeting agent is selected from the group consisting of antibodies, antibody fragments, oligopeptides, enzymes, oligonucleotides, DNA vectors, RNA vectors, aptamers, and lectins.

17. The complex of claim 15, wherein the complex has the following formula: 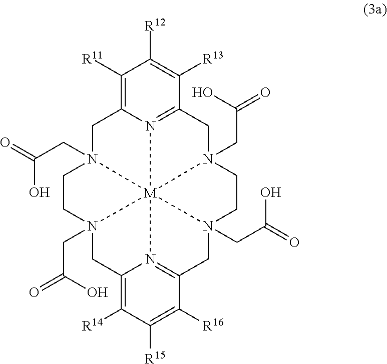
wherein R¹¹, R¹², R¹³, R¹⁴, R¹⁵, R¹⁶, and M are as defined in claim 15.

18. The complex of claim 17, wherein the biological targeting agent is selected from the group consisting of antibodies, antibody fragments, oligopeptides, enzymes, oligonucleotides, DNA vectors, RNA vectors, aptamers, and lectins.

19. A method of treating cancer in a subject, the method comprising administering to said subject a pharmaceutically effective amount of a macrocycle-radionuclide complex having the following formula: 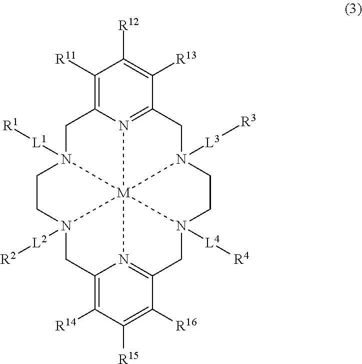
wherein:
L¹, L², L³, and L⁴ are independently selected from a linker of the formula (CH₂)ₚ, wherein p is an integer of 0, 1, 2, or 3;
R¹, R², R³, and R⁴ are independently selected from: (i) oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt; (ii) aryl groups; and (iii) heteroaryl groups;
R¹¹, R¹², R¹³, R¹⁴, R¹⁵, and R¹⁶ are independently selected from: (i) H; (ii) hydrophilic groups of the formula L^{a}_{z}-R^{a}, wherein L^{a} is a hydrocarbon linker containing 1-12 carbon atoms, z is 0 or 1, and R^{a} is independently selected from the group consisting of —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂; and (iii) cancer cell targeting groups of the formula L^{c}_{z}-R^{c}, wherein L^{c} is a hydrocarbon linker containing 1-12 carbon atoms and optionally containing one or more heteroatoms selected from O, N, and S, or L^{c} is a linking particle; z is 0 or 1; and R^{c} is a cancer cell targeting agent; wherein at least one of R¹¹, R¹², R¹³, R¹⁴, R¹⁵, and R¹⁶ is a targeting group L^{c}_{z}-R^{c}; and
M is a radionuclide atom useful in cancer therapy.

20. The method of claim 19, wherein the radionuclide atom is selected from the group consisting of ¹⁷⁷Lu³⁺, ⁴⁷Sc³⁺, ⁹⁰Y³⁺, ¹³⁵La³⁺, ¹⁵²Dy³⁺, ¹⁴⁹Tb³⁺, ¹⁶¹Tb³⁺, ¹⁸⁸Re⁺¹, ¹⁸⁶Re⁺¹, ²¹³Bi³⁺, ²¹²Bi³⁺, ²¹¹At⁺, ²²⁷Th⁴⁺, ²²⁶Th⁴⁺, ²²⁵Ac³⁺, ²²³Ra²⁺, ²²⁴Ra²⁺, ²¹²Pb²⁺, ²¹²Pb⁴⁺, ²⁵⁵Fm³⁺, ²³⁰U⁶⁺, ²³⁰U⁴⁺, ²⁰¹Tl, ¹⁴⁹Pm³⁺, ²³⁹Np⁴⁺, ²³⁹Np⁵⁺, ¹¹⁹Sb³⁺, ¹¹⁹Sb⁵⁺, ⁶⁷Cu, ¹⁵³Sm, and ¹⁶⁶Ho

21. A method of imaging biological tissue in a subject, the method comprising administering to said subject a pharmaceutically effective amount of a macrocycle-radionuclide complex having the following formula: 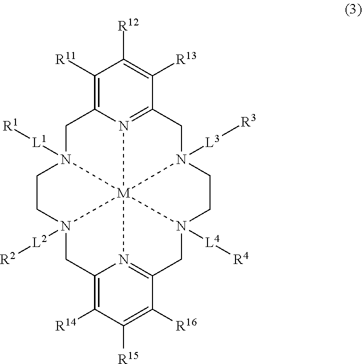
wherein:
L¹, L², L³, and L⁴ are independently selected from a linker of the formula (CH₂)ₚ, wherein p is an integer of 0, 1, 2, or 3;
R¹, R², R³, and R⁴ are independently selected from: (i) oxygen-containing groups independently selected from the group consisting of —COOR′, —OR′, —(OCH₂CH₂)ₙX, —C(O)NHR′, —C(O)N(OH)R′, —P(O)(OH)R, —P(O)(OH)₂, —OS(O)₂OH, —S(O)₂OH, —SO₂R′, S(O)R′, and SO₂NR′₂, wherein R is a hydrocarbon group containing 1-3 carbon atoms, R′ is selected from H and R, n is an integer of 1, 2, or 3, and X is H or OH, and wherein any of the shown H atoms attached to an O atom may be replaced with a monovalent cation to result in a salt; (ii) aryl groups; and (iii) heteroaryl groups;
R¹¹, R¹², R¹³, R¹⁴, R¹⁵, and R¹⁶ are independently selected from: (i) H; (ii) hydrophilic groups of the formula L^{a}_{z}-R^{a}, wherein L^{a} is a hydrocarbon linker containing 1-12 carbon atoms, z is 0 or 1, and R^{a} is independently selected from the group consisting of —COOR′, —C(S)OR′, —OR′, —(OCH₂CH₂)ₙX, —NHR′, —C(O)NR′₂, —C(S)NHR′, —NR′C(O)R′, —NR′C(O)NR′₂, —NR′C(S)R′, —C(O)N(OH)R′, —CN, —NO₂, —P(O)(OR′)R, —P(O)(OR′)₂, —P(O)R′₂, —OS(O)₂OH, —S(O)₂OR′, —SO₂R′, S(O)R′, and SO₂NR′₂; and (iii) cancer cell targeting groups of the formula L^{c}_{z}-R^{c}, wherein L^{c} is a hydrocarbon linker containing 1-12 carbon atoms and optionally containing one or more heteroatoms selected from O, N, and S, or L^{c} is a linking particle; z is 0 or 1; and R^{c} is a cancer cell targeting agent; wherein at least one of R¹¹, R¹², R¹³, R¹⁴, R¹⁵, and R¹⁶ is a targeting group L^{c}_{z}-R^{c}; and
M is a radionuclide atom useful in diagnostic imaging;
followed by imaging of cancerous tissue in the subject.

22. The method of claim 21, wherein the radionuclide atom is selected from the group consisting of ⁴³Sc³⁺, ⁴⁴Sc³⁺, ⁶⁶Ga³⁺, ⁶⁷Ga³⁺, ⁶⁸Ga³⁺, ⁸⁶Y³⁺, ⁸⁸Y³⁺, ⁸⁹Zr⁴⁺, ^{99m}Tc⁺¹, ¹¹¹In³⁺, ^{110m}In³⁺, ¹³²La³⁺, ¹³⁴La³⁺, ¹³⁴Ce³⁺, ¹³⁷Ce³⁺, ¹⁵²Dy³⁺, ¹⁴⁹Tb³⁺, ¹⁵²Tb³⁺, ¹⁵⁵Tb³⁺, ²²⁶Ac³⁺, ¹³¹Ba²⁺, ^{133m}Ba²⁺, ⁹⁰Nb, ¹¹⁷Sb³⁺, ¹¹⁷Sb⁵⁺, ⁵²⁹Mn, ⁶⁴Cu, and ⁴⁵Ti⁴⁺.
